SEQUENCE LISTING (55 sequences)

SEQ ID NO: 1 - DNA, 20 bp, Artificial sequence
  Single strand DNA oligonucleotide
gcagcatgaggattgcacta

SEQ ID NO: 2 - DNA, 20 bp, Artificial sequence
  Single strand DNA oligonucleotide
catgaggctggggaatgtct

SEQ ID NO: 3 - DNA, 20 bp, Artificial sequence
  Single strand DNA oligonucleotide
cctgtcatcagggcaagtga

SEQ ID NO: 4 - DNA, 20 bp, Artificial sequence
  Single strand DNA oligonucleotide
tcgaggcagacgccttattc

SEQ ID NO: 5 - DNA, 20 bp, Artificial sequence
  Single strand DNA oligonucleotide
gtgcattcaacggaacccag

SEQ ID NO: 6 - DNA, 20 bp, Artificial sequence
  Single strand DNA oligonucleotide
agggagctttttcaagcggt

SEQ ID NO: 7 - DNA, 21 bp, Artificial sequence
  Single strand DNA oligonucleotide
ccaagctccaaaccatgttgt

SEQ ID NO: 8 - DNA, 21 bp, Artificial sequence
  Single strand DNA oligonucleotide
acagattgcatgtactccgct

SEQ ID NO: 9 - DNA, 20 bp, Artificial sequence
  Single strand DNA oligonucleotide
accacagtccatgccatcac

SEQ ID NO: 10 - DNA, 21 bp, Artificial sequence
  Single strand DNA oligonucleotide
caccaccctgttgctgtagcc

SEQ ID NO: 11 - DNA, 20 bp, Artificial sequence
  Single strand DNA oligonucleotide
aatttgccaagctcctgaag

SEQ ID NO: 12 - DNA, 20 bp, Artificial sequence
  Single strand DNA oligonucleotide
cgtttggctgaataccttcc

SEQ ID NO: 13 - DNA, 20 bp, Artificial sequence
  Single strand DNA oligonucleotide
tcccaaacaccagtttcctc

SEQ ID NO: 14 - DNA, 20 bp, Artificial sequence
  Single strand DNA oligonucleotide
tgtgtctgcattgctccttc

SEQ ID NO: 15 - DNA, 21 bp, Artificial sequence
  Single strand DNA oligonucleotide
ttgtttcgtaacacagcattg

SEQ ID NO: 16 - DNA, 21 bp, Artificial sequence
  Single strand DNA oligonucleotide
ggcactagtatctttggctga

SEQ ID NO: 17 - DNA, 21 bp, Artificial sequence
  Single strand DNA oligonucleotide
cctacaacaagctgaaaacca

SEQ ID NO: 18 - DNA, 21 bp, Artificial sequence
  Single strand DNA oligonucleotide
ggatagcacagcacaagatca

SEQ ID NO: 19 - DNA, 21 bp, Artificial sequence
  Single strand DNA oligonucleotide
agcagggtctgagaatgaaat

SEQ ID NO: 20 - DNA, 21 bp, Artificial sequence
  Single strand DNA oligonucleotide
gccattgattttgtggttatg

SEQ ID NO: 21 - DNA, 23 bp, Artificial sequence
  Single strand DNA oligonucleotide
ttctacaagatgatcaaggaagg

SEQ ID NO: 22 - DNA, 20 bp, Artificial sequence
  Single strand DNA oligonucleotide
agaattgatccgcacagaat

SEQ ID NO: 23 - DNA, 20 bp, Artificial sequence
  Single strand DNA oligonucleotide
acgaggtccgctacgtaaat

SEQ ID NO: 24 - DNA, 24 bp, Artificial sequence
  Single strand DNA oligonucleotide
ggctctccagacattttacatatt

SEQ ID NO: 25 - DNA, 21 bp, Artificial sequence
  Single strand DNA oligonucleotide
aaagaagtcgcagacgaagga

SEQ ID NO: 26 - DNA, 21 bp, Artificial sequence
  Single strand DNA oligonucleotide
tattggatggtggcattttca

SEQ ID NO: 27 - protein, 144 aa, homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 28 - protein, 271 aa, homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 29 - protein, 271 aa, homo sapiens; identical to SEQ ID NO: 28

SEQ ID NO: 30 - protein, 269 aa, homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 31 - protein, 212 aa, homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 32 - protein, 136 aa, homo sapiens
MCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 33 - protein, 189 aa, homo sapiens
MNSFSTIPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 34 - protein, 211 aa, homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 35 - protein, 228 aa, homo sapiens
MQSLPNSNGAAAGRDFKMKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 36 - protein, 202 aa, homo sapiens
MQSLPNSNGAAAGRDFKMKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 37 - protein, 159 aa, homo sapiens
MPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 38 - protein, 185 aa, homo sapiens
MKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 39 - protein, 88 aa, homo sapiens
MKVLAAVHSPGGAVPQQPGQAMWPQRDGLPALPRQRHGEGQAGGAVPHSRVPWHLPGQHHPGPEDPQPQCPQPPQQAQRHRRHPARPP

SEQ ID NO: 40 - protein, 202 aa, homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 41 - protein, 288 aa, homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 42 - protein, 155 aa, homo sapiens [sequence omitted]

SEQ ID NO: 43 - protein, 1166 aa, homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 44 - protein, 1165 aa, homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 45 - protein, 1207 aa, homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 46 - protein, 1010 aa, homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWGQCNQLHGGRSPSYVEWAQSKAAGFQYPVIRAPVPR

SEQ ID NO: 47 - protein, 233 aa, homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 48 - DNA, 24 bp, Artificial sequence
  Single strand DNA oligonucleotide
tgtccgtggttgccaaggataaca

SEQ ID NO: 49 - DNA, 24 bp, Artificial sequence
  Single strand DNA oligonucleotide
aatccgggtacctgcttgtgagtt

SEQ ID NO: 50 - DNA, 24 bp, Artificial sequence
  Single strand DNA oligonucleotide
aacccaacaagtggcccaagatac

SEQ ID NO: 51 - DNA, 24 bp, Artificial sequence
  Single strand DNA oligonucleotide
ctttggacactccagctctgtcat

SEQ ID NO: 52 - DNA, 24 bp, Artificial sequence
  Single strand DNA oligonucleotide
agtattcatggtgatcgggagcag

SEQ ID NO: 53 - DNA, 24 bp, Artificial sequence
  Single strand DNA oligonucleotide
gcaacaagaactgggcactttcca

SEQ ID NO: 54 - DNA, 21 bp, Artificial sequence
  Single strand DNA oligonucleotide
agcttgaaatacgtggccaga

SEQ ID NO: 55 - DNA, 22 bp, Artificial sequence
  Single strand DNA oligonucleotide
tgagcagttcacacttcatccc

(19) United States
(12) Patent Application Publication
(10) Pub. No.: US 2023/0064363 A1
(43) Pub. Date: Mar. 2, 2023

(54) MATURATION OF SPERMATOGONIAL CELLS IN VITRO BT GROWTH FACTORS AND HORMONES

(71) Applicant: B.G. Negev Technologies & Applications Ltd., at Ben-Gurion University, Beer-Sheva (IL)

(72) Inventor: Mahmoud HULEIHEL, Beer-Sheva (IL)

(73) Assignee: B.G. Negev Technologies & Applications Ltd., at Ben-Gurion University, Beer-Sheva (IL)

(21) Appl. No.: 17/799,948

(22) PCT Filed: Feb. 16, 2021
(86) PCT No.: PCT/IL2021/050179

§ 371 Date: Aug. 16, 2022

Related U.S. Application Data
(60) Provisional application No. 62/977,293, filed on Feb. 16, 2020.
(60) Provisional application No. 63/124,820, filed on Dec. 13, 2020.

(51) Int. Cl. C12N 5/00; C12N 5/076; A61K 31/675; A61K 35/52; A61P 15/00

(52) U.S. Cl. CPC C12N 5/0068; C12N 5/061; A61K 31/675; A61K 35/52; A61P 15/00; C12N 2501/392; C12N 2513/00; C12N 2533/78; C12N 2501/2301; C12N 2501/2306; C12N 2501/22

(57) ABSTRACT

Provided are methods of in vitro maturation of spermatogonium by culturing the spermatogonium in a three-dimensional methylcellulose culture system (MCS) in a culture medium which comprises an effective concentration of a factor selected from the group consisting of granulocyte macrophages-colony stimulating factor (GM-CSF), interleukin-1 alpha (IL-1alpha), interleukin-1 beta (IL-1beta) and interleukin-6 (IL-6), under conditions capable of differentiating the spermatogonium into at least meiotic and/or postmeiotic cells, thereby in vitro maturing the spermatogonium.

### RELATED APPLICATION/S

This application claims the benefit of priority of U.S. Provisional Patent Application No. 62/977,293 filed on Feb. 16, 2020 and U.S. Provisional Patent Application No. 63/124,820 filed on Dec. 13, 2020, the contents of which are incorporated herein by reference in their entirety.

### SEQUENCE LISTING STATEMENT

The ASCII file, entitled 86551 SequenceListing.txt, created on 16 Feb. 2021, comprising 77,119 bytes, submitted concurrently with the filing of this application is incorporated herein by reference.

### FIELD AND BACKGROUND OF THE INVENTION

The present invention, in some embodiments thereof, relates to a method of in vitro maturation of spermatogonium and, more particularly, but not exclusively, to a method of treating a subject in need of mature sperm cells.

Spermatogenesis, a process of spermatogonial cell development to meiotic and postmeiotic stages in the testis, is a cyclical process which occurs within the seminiferous tubules. Spermatogonial stem cells (SSCs) are located close to the basement membrane of the seminiferous tubule; they proliferate, differentiate and undergo meiosis and spermiogenesis to generate mature sperm [Huleihel, M et al. Growth Factors 25, 236-252 (2007); Huleihel, M., et al. Asian J Androl 17, 972-980 (2015); Michailov, Y., et al. Leuk Lymphoma 5, 1126-1135 (2019)]. Spermatogenesis is controlled by both endocrine and paracrine/autocrine factors, including for example systems luteinizing hormone (LH), follicle stimulating hormone (FSH), testosterone, IL-1α, TNF-α.

Childhood cancer is estimated to affect 0.1% of prepubertal boys up to 15 years of age [Aslam I, et al. Hum Reprod. 2000 October; 15(10):2154-9]. Among those, acute lymphoblastic leukemia (ALL) affects around 26% of the patients. About 80% will survive the disease due to the progress in anti-cancer treatments [Desandes E. et al. Cancer Treat Rev (2007) 33:609-615; Hudson M M. Obstet Gynecol (2010) 116: 1171-11831 Ward E, et al. CA Cancer J Clin (2014) 64:83-103]. These anti-cancer treatments are mostly gonadotoxic (chemotherapy and/or radiotherapy), which in some cases, are a combination of different types of chemotherapeutic agents or a combination with radiotherapy and may lead to impairment of fertility and even to permanent azoospermia. This depends on the type of dose and combination of anti-cancer treatment agents [Kliesch S, et al. Med Pediatr Oncol. (1996) 26(1):20-7]. These agents may affect both spermatogenic and testicular somatic cells. However, some adolescent patients (16-68%) will become azoospermic following chemo-/radiotherapy [Kliesch S, et al. Med Pediatr Oncol. (1996) 26(1):20-7]. For example, cyclophosphamide (CP), an alkylating agent from nitrogen mustard family, is commonly used for treatment of various cancers [Calabresi, P., Parks, R. Antiproliferative agents and drugs used for immunosuppression (ed. Goodman-Gillman, A., Goodman, L. S. & Gillman, A.) 1256-1306 (Macmillan, 1980)] and has diverse side effects on male fertility. It was found that exposure of mouse fetus to CP reduced their testes weights, induced testicular cancer, caused seminiferous tubules atrophy and impaired their spermatogenesis [Comish, P. B. PloS one 9, e93311 (2014)]. Treatment of mouse and human with CP lead to impairment of spermatogenesis including the development of oligospermia and even long or permanent azoospermia [Elangovan, N., et al; Toxicology 222, 60-70 (2006); Codrington, A. M., et al. J Androl 25, 354-362 (2004)]. It also may affect sperm DNA structure and may lead to embryonic loss [Codrington, A. M., et al. Hum Reprod 22, 1431-1442 (2007); Arnon, J., et al. Hum Reprod Update 7, 394-403 (2001)]. An in vitro study revealed that CP inhibited telomerase activity in mice spermatogonial germ cells [Liu, M., et al. Biology of reproduction 90, 72-1 (2014)] and led to histological alternation of the seminiferous tubules [Kanth, M. A., et al. Indo American Journal of Pharmaceutical Research 4, 2645-2649 (2014)].

Fertility preservation of adult cancer patients is possible by cryopreservation of their sperm before chemotherapy/radiotherapy treatment. However, since prepubertal males do not produce spermatozoa, sperm cell cryopreservation for fertility preservation is unfeasible in this age group. The only suggested possibilities for their fertility preservation are testicular tissue or cell cryopreservation before aggressive anti-cancer treatments, for future use as autotransplantation, or in vitro maturation of their spermatogonial stem cells (SSCs) to sperm (Gassei K, et al. Fertil Steril. (2016) 105(2):256-66; Picton H M, et al. Hum Reprod. (2015) 30(11):2463-75; Mahmoud H. Stem Cells. (2012) 30(11):2355-60; Jahnukainen K, et al. J Clin Endocrinol Metab. (2012) 97(12):4341-51). The feasibility and safety of fertility preservation in prepubertal cancer patient boys via cryopreservation of testicular biopsies has been reported [Ginsberg J P, et al. Hum Reprod (2010) 25: 37-41; Wyns C, et al. Hum Reprod (2011) 26: 337-347; Wyns C, et al. Hum Reprod (2015) 30: 2022-2030]. Also, it was shown that testicular growth of the biopsied testis was similar to the non-biopsied contralateral testis until one year after surgery [Uijldert M, et al. Hum Reprod (2017) 32: 2366-2372]. The limitation of using testicular tissue or cells for autotransplantation is the possibility of presence of residual cancer cells which may restore the disease. For instance, microinjection of rat T-cell leukemia (around 20 leukemia cells) mixed with germ cells into rat testis resulted in a cancer relapse [Jahnukainen K, et al. Cancer Res (2001) 61: 706-710]. Today, there is no safe methodology to isolate cancer cells from testicular tissue of cancer patients [Fujita K, et al. Cancer Res 2006; 66:11166-71; Geens M, et al. Fertil Steril 2011; 787:91-95; Dovey S L et al. J Clin Invest 2013; 123:1833-43]. On the other hand, intratesticular transplantation of mouse spermatogonial stem cells that were grown in vitro into busulfan-treated mice did not affect cancer incidence or the long-term survival rate compared to non-transplanted busulfan-treated mice [Mulder C L, et al. Hum Reprod (2018) 33: 81-90]. Additionally, significant limitation of this approach is the scarce number of SSCs present in the testicle relatively to other germ cell population. In adult mouse testes, this was estimated to be 0.03% [Nagano M, et al. Biol Reprod 2003; 68: 2207-2214], the biopsies obtained are very small, and spermatogonial cells comprised about 3% of the cell population of testicular biopsies from prepubertal boys [Wu X, et al. Proc. Nat. Acad. Sci (2009) 106: 21672-21677]. Considering the small volume of the biopsy, it can be realized that the number of SSCs would be extremely low.

If successful, in vitro differentiation of SSCs to sperm is one of the safe options for fertility preservation. Recently, the capacity of induction propagation of human SSCs from adult and prepubertal boys was demonstrated [Sadri-Anlekani H, et al. J Am Med Asso (2011) 305: 2416-2418; Sadri-Ardekani H, et al. J Am Med Asso (2009) 19:2127-2134]. In an animal model, Sato et al., using organ culture of testis from immature mouse demonstrated the capacity of induction SSCs to meiotic and postmeiotic stages including the generation of fertile sperm in vitro [Sato T, et al. Nature (2011) 471:504-507]. In this system, the microenvironment niches of the SSCs and the cellular interactions in the seminiferous tubule and in the interstitial compartments remained intact, and thus enabled proliferation and differentiation of the SSCs. In-vitro proliferation and development of isolated spermatogonial cells to meiotic and postmeiotic stages including sperm like cells using three-dimension (3D) in vitro culture systems have been disclosed in e.g. Abu Elhija M, et al. Asian J Androl (2012) 14:285-293; Stukenborg J-B, et al. Mol Hum Reprod (2009) 15: 521-529; Stukenborg J B et al. J Androl. (2008) May-June; 29(3):312-29; Huleihel M, et al. Asian J Androl (2015) 17:972-980; Abofoul-Azab, M et al. (2018) Stem Cells Dev. 1; 27(15):1007-1020); Abofoul-Azab, M. et al. (2019) Int. J. Mol. Sci. 20: 470; doi:10.3390/ijms20030470; and AbuMadighem, A., et al. (2018) Int. J. Mol. Sci. 19: 3804-3820).

Additional background art include:
Huleihel, M., et al. Growth Factors (2007) 25(4): 236-252;
Huleihel, M. et al. Asian J. Androl. (2004) 6: 259-268;
Michailov, Y. et al. Leuk & Lymph. (2018) 1-10. doi: 10.1080/10428194.2018.1533126;
Ghenima Dirami, et al. Biology of reproduction (1999). 61(1): 225-230;
Lourdes, T. et al. Theriogenology (2003). 60(6): 1083-1095;
Zambrano, A. et al. Journal of cellular biochemistry (2001) 80(4): 625-634.

### SUMMARY OF THE INVENTION

According to an aspect of some embodiments of the present invention there is provided a method of in vitro proliferation and/or maturation of spermatogonium, comprising culturing the spermatogonium in a three-dimensional methylcellulose culture system (MCS) in a culture medium which comprises an effective concentration of a factor selected from the group consisting of granulocyte macrophages-colony stimulating factor (GM-CSF), interleukin-1 alpha (IL-1α), interleukin-1 beta (IL-1β) and interleukin-6 (IL-6), under conditions capable of inducing proliferation of the spermatogonium and/or differentiating the spermatogonium into at least meiotic and/or postmeiotic cells, thereby in vitro proliferating and/or maturing the spermatogonium. According to an aspect of some embodiments of the present invention there is provided a method of in vitro proliferation and/or maturation of spermatogonium, comprising culturing the spermatogonium in a three-dimensional methylcellulose culture system (MCS) in a culture medium which comprises an effective concentration of a granulocyte macrophages-colony stimulating factor (GM-CSF) and/or an effective concentration of interleukin-1 alpha (IL-1α) under conditions capable of inducing proliferation of the spermatogonium and/or differentiating the spermatogonium into at least meiotic and/or postmeiotic cells, thereby in vitro proliferating and/or maturing the spermatogonium.

According to an aspect of some embodiments of the present invention there is provided a method of in vitro maturation of spermatogonium, comprising culturing the spermatogonium in a three-dimensional methylcellulose culture system (MCS) in a culture medium which comprises an effective concentration of a granulocyte macrophages-colony stimulating factor (GM-CSF) and/or an effective concentration of interleukin-1 alpha (IL-1α) under conditions capable of differentiating the spermatogonium into postmeiotic cells, thereby in vitro maturing the spermatogonium.

According to an aspect of some embodiments of the present invention there is provided a method of in vitro proliferation and/or maturation of spermatogonium, comprising culturing the spermatogonium in a three-dimensional methylcellulose culture system (MCS) in a culture medium which comprises Sertoli cells conditioned medium under conditions capable of inducing proliferation of the spermatogonium and/or differentiating the spermatogonium into at least meiotic and/or postmeiotic cells, thereby in vitro proliferating and/or maturing the spermatogonium.

According to an aspect of some embodiments of the present invention there is provided a method of in vitro maturation of spermatogonium, comprising culturing the spermatogonium in a three-dimensional methylcellulose culture system (MCS) in a culture medium which comprises Sertoli cells conditioned medium under conditions capable of differentiating the spermatogonium into postmeiotic cells, thereby in vitro maturing the spermatogonium.

According to an aspect of some embodiments of the present invention there is provided a cell obtainable according to the method.

According to some embodiments of the invention, the cell is characterized by at least the expression of acrosin.

According to an aspect of some embodiments of the present invention there is provided a cell obtainable according to the method of some embodiments of the invention wherein the cell is characterized by at least the expression of acrosin.

According to an aspect of some embodiments of the present invention there is provided a method of treating a subject in need of mature sperm cells, comprising:

(a) obtaining a spermatogonium from the subject, and

(b) subjecting the spermatogonium to an in vitro maturation according to the method of some embodiments of the invention,

thereby generating mature sperm cells of the subject, and treating the subject.

According to some embodiments of the invention, the method further comprising culturing cells developed following a pre-determined time period from the spermatogonium in a second culture medium which comprises the factor and an effective concentration of testosterone.

According to some embodiments of the invention, the method further comprising culturing cells developed following a pre-determined time period from the spermatogonium in a second culture medium which comprises the GM-CSF and/or the IL-1a and an effective concentration of testosterone.

According to some embodiments of the invention, the predetermined time period is two weeks±2 days.

According to some embodiments of the invention, the culturing in the second culture medium is effected for a second predetermined time period.

According to some embodiments of the invention, the second predetermined time period is two weeks±2 days.

According to some embodiments of the invention, the culture medium further comprises an effective concentration of at least one growth factor selected from the group consisting of Glial cell line-derived neurotrophic factor (GDNF), leukemia inhibitory factor (LIF), basic fibroblast growth factor (bFGF), and epidermal growth factor (EGF).

According to some embodiments of the invention, the second culture medium further comprises an effective concentration of at least one growth factor selected from the group consisting of Glial cell line-derived neurotrophic factor (GDNF), leukemia inhibitory factor (LIF), basic fibroblast growth factor (bFGF), and epidermal growth factor (EGF).

According to some embodiments of the invention, the culture medium comprises serum replacement.

According to some embodiments of the invention, the second culture medium comprises serum replacement.

According to some embodiments of the invention, the culture medium comprises STEM PRO® (Thermo Fisher Scientific) supplement.

According to some embodiments of the invention, the second culture medium comprises STEM PRO® (Thermo Fisher Scientific) supplement.

According to some embodiments of the invention, the culture medium further comprises a Sertoli cell conditioned medium.

According to some embodiments of the invention, the culture medium further comprises at least one growth factor selected from the group consisting of: Glial cell line-derived neurotrophic factor (GDNF), leukemia inhibitory factor (LIF), and stem cell factor (SCF).

According to some embodiments of the invention, the culture medium further comprises an effective concentration of a factor selected from the group consisting of granulocyte macrophages-colony stimulating factor (GM-CSF), interleukin-1 alpha (IL-1α), interleukin-1 beta (IL-1β) and interleukin-6 (IL-6).

According to some embodiments of the invention, the culture medium further comprises an effective concentration of a granulocyte macrophages-colony stimulating factor (GM-CSF) and/or an effective concentration of interleukin-1 alpha (IL-1α).

According to some embodiments of the invention, the culture medium comprises serum replacement.

According to some embodiments of the invention, the Sertoli condition medium comprises of 10%-40% (volume/volume) of the culture medium.

According to some embodiments of the invention, the spermatogonium is comprised in a testicular biopsy of the subject.

According to some embodiments of the invention, the testicular biopsy is obtained from a prepubertal male subject.

According to some embodiments of the invention, the testicular biopsy is obtained from a non-obstructive azoospermic patient.

According to some embodiments of the invention, the testicular biopsy is obtained from a human male which has been subjected to an aggressive chemotherapy and/or an aggressive radiotherapy.

According to some embodiments of the invention, the method comprising obtaining the spermatogonium from a subject prior to the culturing.

According to some embodiments of the invention, the spermatogonium is of a subject who is infertile or at risk of becoming infertile.

According to some embodiments of the invention, the spermatogonium is of a subject who is a non-obstructive azoospermic subject.

According to some embodiments of the invention, the spermatogonium if of a subject who is a prepubertal subject.

According to some embodiments of the invention, the spermatogonium if of a subject in need of aggressive chemotherapy and/or aggressive radiotherapy.

According to some embodiments of the invention, the spermatogonium is of subject diagnosed with cancer.

According to some embodiments of the invention, the spermatogonium is of a subject diagnosed with autoimmune disease.

According to some embodiments of the invention, the spermatogonium if of a subject diagnosed with thalassemia.

According to some embodiments of the invention, the spermatogonium is of a subject in need of bone marrow transplantation.

According to some embodiments of the invention, the spermatogonium is of a subject inflicted with cancer associated with male infertility.

According to some embodiments of the invention, the method further comprises identifying a meiotic cell, a postmeiotic cell and/or a mature sperm cell following the culturing in vitro.

According to some embodiments of the invention, the method further comprises identifying a meiotic cell and/or a postmeiotic cell following the culturing in vitro.

According to some embodiments of the invention, the method further comprises identifying a cell expressing acrosin following the culturing in vitro.

According to some embodiments of the invention, the method comprising isolating a mature cell following the culturing in vitro, wherein the mature cell is characterized by at least the expression of acrosin.

According to some embodiments of the invention, the subject is a prepubertal male subject.

According to some embodiments of the invention, the prepubertal male subject is in need of an aggressive chemotherapy and/or an aggressive radiotherapy.

According to some embodiments of the invention, the prepubertal male subject has been subjected to an aggressive chemotherapy and/or an aggressive radiotherapy.

According to some embodiments of the invention, the prepubertal male subject is diagnosed with cancer.

According to some embodiments of the invention, the prepubertal male subject is diagnosed with autoimmune disease.

According to some embodiments of the invention, the prepubertal male subject is diagnosed with thalassemia.

According to some embodiments of the invention, the prepubertal male subject is in need of bone marrow transplantation.

According to some embodiments of the invention, the cancer comprises a hematological cancer.

According to some embodiments of the invention, the cancer is acute myeloid leukemia (AML).

According to some embodiments of the invention, the cancer comprises a solid tumor.

According to some embodiments of the invention, the cancer is selected from the group consisting of brain tumor, extragonadal germ-cell tumor (EGGCTs), sarcoma, testicular germ cell tumor (TGCT), prostate cancer, skin cancer, Hodgkin lymphoma (HL), and non-Hodgkin lymphoma (NH L).

According to some embodiments of the invention, the subject is a non-obstructive azoospermic patient.

According to some embodiments of the invention, the subject is in need of aggressive chemotherapy and/or aggressive radiotherapy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C demonstrate the effects of GM-CSF and testosterone on development of premeiotic cells. Isolated seminiferous tubule cells were cultured in methylcellulose culture system (MCS, 42%) in a medium comprising KnockOut serum replacement (KSR), StemPro, EGF, FGF, LIF and GDNF (control medium) for two weeks followed by addition of fresh media and culturing for additional two weeks. When indicated, the culture medium was further supplemented with GM-CSF and/or testosterone. Pre-cult—isolated cells prior to culturing control; CT—control medium for four weeks; CT-T—control medium for two weeks followed by testosterone supplemented medium for additional two weeks; GM—medium supplemented with GM-CSF for four weeks; GM-T—medium supplemented with GM-CSF for two weeks followed by medium supplemented with GM-CSF and testosterone for additional two weeks. FIG. 1A shows the percentages of VASA positive cells as determined by immunofluorescence staining. FIG. 1B shows VASA RNA expression levels, as compared to CT cultured cells, as determined by qPCR analysis. FIG. 1C shows PLZF RNA expression levels, as compared to CT cultured cells, as determined by qPCR analysis. ### p<0.001 compared to pre-cult; * p<0.05, ** p<0.01, *** p<0.001 compared to control.

FIGS. 2A-B demonstrate the effects of GM-CSF and testosterone on development of meiotic cells. Isolated seminiferous tubule cells were cultured as described in FIGS. 1A-C hereinabove. Pre-cult—isolated cells prior to culturing control; CT—control medium for four weeks; CT-T—control medium for two weeks followed by testosterone supplemented medium for additional two weeks; GM—medium supplemented with GM-CSF for four weeks; GM-T—medium supplemented with GM-CSF for two weeks followed by medium supplemented with GM-CSF and testosterone for additional two weeks. FIG. 2A shows the percentages of BOULE positive cells as determined by immunofluorescence staining. FIG. 2B shows BOULE RNA expression levels, as compared to CT cultured cells, as determined by qPCR. ### p<0.001 compared to pre-cult; * p<0.05, ** p<0.01, *** p<0.001 compared to control.

FIGS. 3A-B demonstrate the effects of GM-CSF and testosterone on development of postmeiotic cells. Isolated seminiferous tubule cells were cultured as described in FIGS. 1A-C hereinabove. Pre-cult—isolated cells prior to culturing control; CT—control medium for four weeks; CT-T—control medium for two weeks followed by testosterone supplemented medium for additional two weeks; GM—medium supplemented with GM-CSF for four weeks; GM-T—medium supplemented with GM-CSF for two weeks followed by medium supplemented with GM-CSF and testosterone for additional two weeks. FIG. 3A shows the percentages of acrosin positive cells as determined by immunofluorescence staining. FIG. 3B shows acrosin RNA expression levels, as compared to CT cultured cells, as determined by qPCR. ## p<0.01 compared to pre-cult; * p<0.05, ** p<0.01, *** p<0.001 compared to control.

FIGS. 4A-F demonstrate that cyclophosphamide (CP) significantly decreases testicular weight and impairs seminiferous tubule normal histology in immature mice. CP (100 mg/kg in 100 μl) or PBS (control, CT, 100 μl) was intraperitoneally injected to immature mice. One to 5 weeks after the last injection, mice were sacrificed, and testes were removed, weighed and fixed in Bouin's solution for histological evaluation. FIG. 4A is a graph demonstrating testes weight following CP treatment (CP) compared to control (Control). FIG. 4B shows representative images of histological evaluation of the seminiferous tubules stained with hematoxylin-eosin (H&E). FIG. 4C is a graph summing seminiferous tubule damage evaluated by histological evaluation after 1-5 weeks post CP (CP) treatment compared to the Control. FIG. 4D shows representative images of histological evaluation of the seminiferous tubules stained with H&E ten days post-treatment. FIG. 4E is a graph demonstrating testes weight ten days following CP treatment (CP) compared to control (CT). FIG. 4F is a graph demonstrating the total number of cells isolated from the seminiferous tubules were counted ten days following CP treatment (CP) compared to control (CT). (FIG. 4B)—×20 light microscope magnification (100 sm scale). (FIG. 4D)—×40 light microscope magnification (100 μm scale). **—p<0.01 and ***—p<0.001.

FIGS. 5A-D demonstrate that treatment of immature mice with CP significantly decreases the number of subpopulations of spermatogenic cells compared to control. CP or PBS (Control) was injected as descried in FIGS. 4A-F. Ten days post-treatment, testes were removed, seminiferous tubules were separated, and cells were enzymatically isolated from the seminiferous tubules. The premeiotic cells that express α-6-integrin, VASA, CD9, GFR-α and c-KIY or the meiotic cells that express the markers BOULE and CREM and the meiotic/postmeiotic cells that express the marker acrosin were identified by immunofluorescence staining using specific antibodies for each cell marker. FIGS. 5A-B show representative staining images following CP treatment and FIGS. 5C-D show graphs summing the number per testis of the identified premeiotic, meiotic and meiotic/postmeiotic cells following CP treatment as compared to control (CT). NC—negative control.

FIGS. 6A-C demonstrate the effect of treatment of immature mice with CP on the number of Sertoli and peritubular cells and on the functionality of Sertoli cells. CP or PBS (Control, Conn.) was injected as descried in FIGS. 4A-F. Ten days post-treatment, testes were removed, seminiferous tubules were separated, and cells were enzymatically isolated from the seminiferous tubules. Sertoli cells and peritubular cells were identified by immunofluorescence staining using specific antibodies for each cell type (vimentin—a marker for Sertoli cells and α-sma—a marker for peritubular cells). FIG. 6A shows representative staining images following CP treatment and FIG. 6B is graph summing the number per testis of the identified marker following CP treatment as compared to control (CT). NC—negative control. FIG. 6C is a graph demonstrating RNA expression levels of factors known to be produced by Sertoli cells [androgen binding protein (ABP), inhibin, FSH-receptor (FSH-R), transferrin, inhibin] in cells isolated from seminiferous tubules of CT or CP-treated mice ten days post-treatment, as determined by qPCR using specific primers.

FIGS. 7A-F demonstrate that isolated cells from seminiferous tubules of CP-treated immature mice develope colonies in vitro in a methylcellulose culture system (MCS). Isolated cells from seminiferous tubules obtained from CP-treated immature mice ten days after the last injection were cultured in a MCS composed of 42% methylcellulose, KSR (10%), StemPro, and growth factors (GDNF, LIF, FGF, EGF) in the absence (control medium) or presence of IL-1α, TNF-α, FSH, testosterone (T) or both IL-1α+T, TNF-α+T, FSH+T. Developed colonies after 4-5 weeks of culture are presented in the different groups demonstrating similar morphology following treatment with the different agents. Scale bar represents 100 μm, magnification ×20.

FIGS. 8A-C demonstrate the effect of IL-1α on the development of spermatogenesis in vitro. Seminiferous tubule cells were isolated from 7-days-old mice by enzymatic digestion. Isolated cells were cultured in methylcellulose culture system (MCS, 42%) in the presence of KSR, StemPro, EGF, FGF, LIF and GDNF in the absence (control medium, CT) or with addition of IL-1α (10 pg/ml) from the beginning of the culture (CT+IL-1) or only after two weeks of the culture for additional two weeks (CT+IL-1 after two weeks). Cells and clusters/colonies developed in the culture were collected after 4 weeks of culture and examined for the presence of the premeiotic cells (VASA, FIG. 8A), or the meiotic cells (BOULE, FIG. 8B) or the Meiotic/postmeiotic cells (ACROSIN, FIG. 8C) by immunofluorescence staining using antibodies specific for each marker.

FIGS. 9A-C demonstrate the effect of IL-1β on the development of spermatogenesis in vitro. Seminiferous tubule cells were isolated from 7-days-old mice by enzymatic digestion. Isolated cells were cultured in methylcellulose culture system (MCS, 42%) in the presence of KSR, StemPro, EGF, FGF, LIF and GDNF in the absence (control medium, CT) or with addition of IL-1β (1, 10 or 100 pg/ml) from the beginning of the culture. Cells and clusters/colonies developed in the culture were collected after 4 weeks of culture and examined for the presence of the premeiotic cells (VASA, FIG. 9A), or the meiotic cells (BOULE, FIG. 9B) or the Meiotic/postmeiotic cells (ACROSIN, FIG. 9C) by immunofluorescence staining using antibodies specific for each marker. * Significant compared to control; $ significant comparison between 10 and 100 pg/ml.

FIGS. 10A-C demonstrate the effect of IL-6 on the development of spermatogenesis in vitro. Seminiferous tubule cells were isolated from 7-days-old mice by enzymatic digestion. Isolated cells were cultured in methylcellulose culture system (MCS, 42%) in the presence of KSR, StemPro, EGF, FGF, LIF and GDNF in the absence (control medium, CT) or with addition of IL-6 (1, 10 or 100 pg/ml) from the beginning of the culture. Cells and clusters/colonies developed in the culture were collected after 4 weeks of culture and examined for the presence of the premeiotic cells (VASA positive cells, FIG. 10A), or the meiotic cells (BOULE positive cells, FIG. 10B) or the Meiotic/postmeiotic cells (ACROSIN positive cells, FIG. 10C) by immunofluorescence staining using antibodies specific for each marker. * Significant compared to control.

### DESCRIPTION OF SPECIFIC EMBODIMENTS OF THE INVENTION

The present invention, in some embodiments thereof, relates to a method of in vitro maturation of human spermatogonium and, more particularly, but not exclusively, to a method of treating a subject in need of mature sperm cells.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Spermatogenesis is a complicated process, which is composed of premeiotic, meiotic and postmeiotic stages. This process is developed in vivo in the seminiferous tubules of adult males. It is regulated by endocrine system and by testicular autocrine and paracrine factors that compose specific microenvironments for each stage of spermatogonial cell (SPGC) development to generate sperm. Recently, fertile sperm were generated from immature mouse using testicular organ culture system. Using three-dimension (3D) in vitro culture system, the development of sperm-like cells from SPGCs of immature mice, and the development of round spermatid from SPGCs of prepubertal monkeys were demonstrated. Induction of the proliferation of SPGCs in vitro from prepubertal cancer patients was also demonstrated (e.g. Sadri-Ardekani H et al. J Am Med Asso (2011) 305: 2416-2418; Sadri-Ardekani H, et al. J Am Med Asso (2009_; 19:2127-2134).

While reducing specific embodiments of the present invention to practice, the present inventor has uncovered that the addition of granulocyte-colony stimulating factors (G-CSF), interleukin-1α (IL-1α), interleukin-1β (IL-1β) or interleukin-6 (IL-6) alone or in combination with testosterone to the culture medium of spermatogonial cells obtained from immature mice leads to proliferation of premeiotic cells and the development of meiotic and postmeiotic cells in a three-dimensional (3D) methylcellulose culture system (MCS).

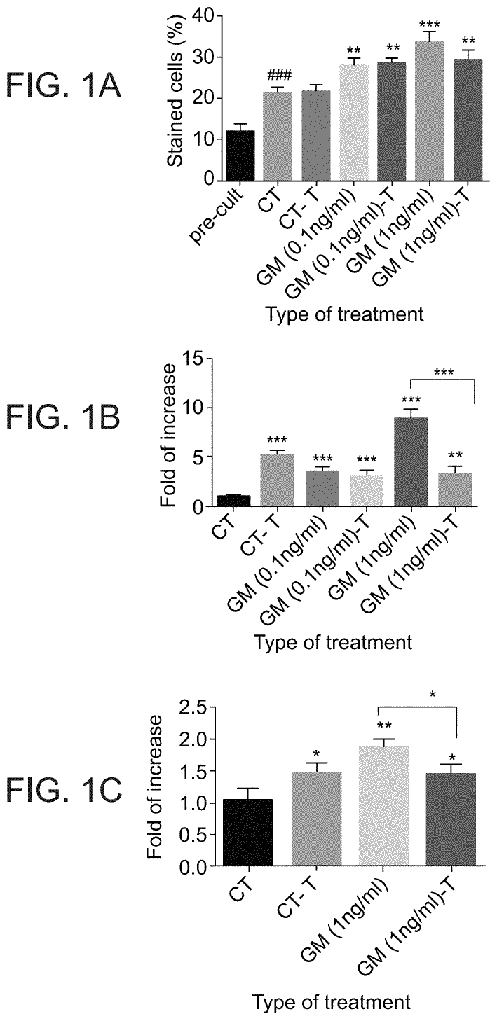

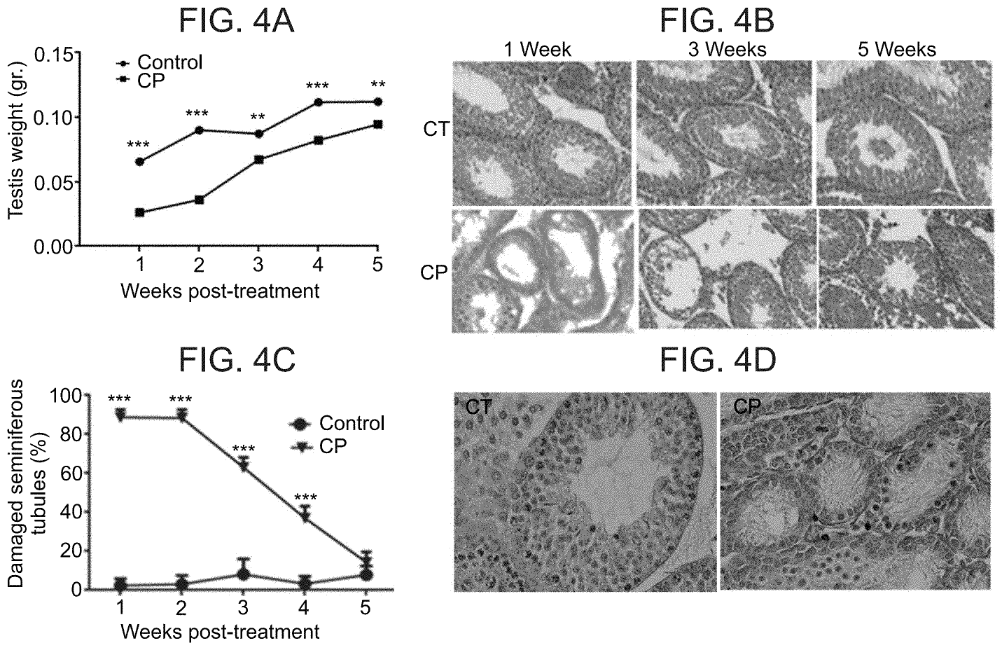

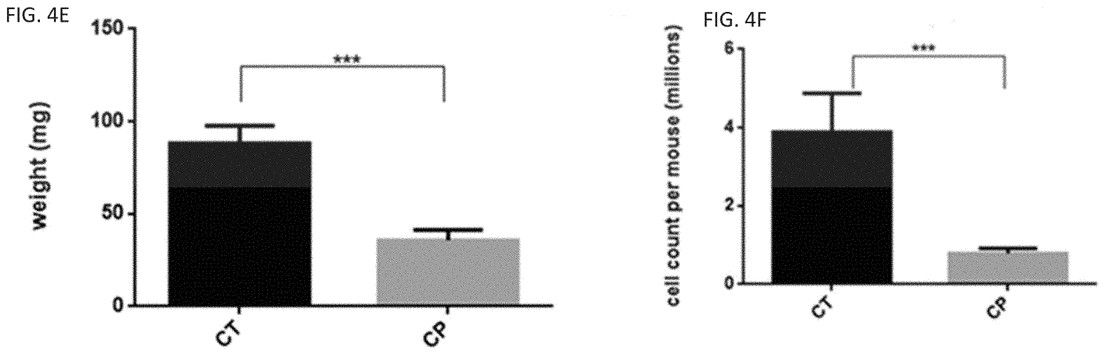

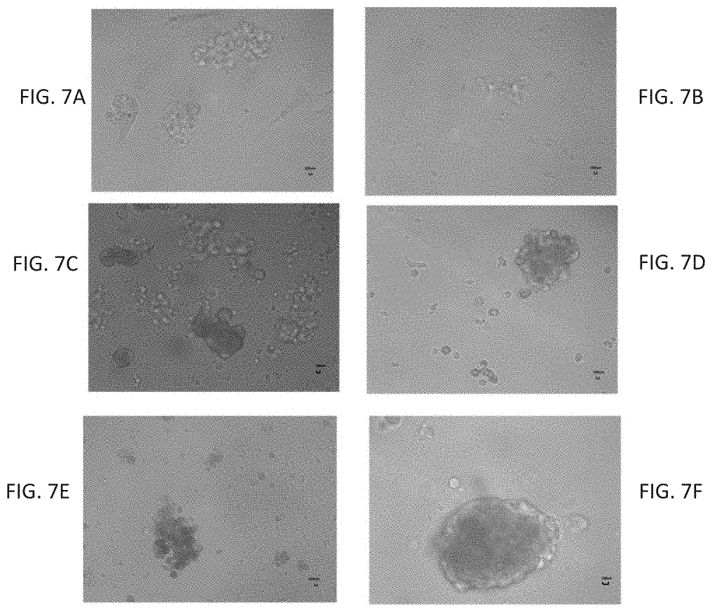

As is illustrated hereinunder and in the examples section, which follows, the present inventor shows that addition of G-CSF, IL-1α, IL-1β or IL-6 alone or in combination with testosterone to the culture medium of spermatogonial cells obtained from immature (i.e. prepubertal) mice leads to proliferation of the premeiotic cells and differentiation into meiotic and postmeiotic stages including the generation of sperm-like cells in a three-dimensional methylcellulose culture system (MCS) (Examples 1 and 3-5, FIGS. 1A-3B and 8A-10C). In addition, the present inventor was able to show, for the first time, that cyclophosphamide (CP) treatment of immature mice decreased testicular weight, impaired spermatogenesis and significantly decreased the number of subpopulations of spermatogonial cells, Sertoli and peritubular cells and also affected the functionality of Sertoli cells (Example 2 FIG. 4A-6C). However, the present inventor was able to induce the proliferation of spermatogonial cells from the CP-treated immature mice and increase their differentiation to meiotic and postmeiotic stages in vitro in the presence of several cytokines and hormones in a 3D MCS (Example 2, Table 1 and FIGS. 7A-F).

Finding factors and processes that could induce spermatogenesis in vitro may help in developing future therapeutic strategies for infertile male subjects or male subjects at risk of becoming infertile.

Specifically, the presence of spermatogonial cells (SPGCs) in the testes of e.g. prepubertal males [e.g. prepubertal cancer patient boys (PCPBs]), non-obstructive azzospermic patients or patients having a disease associated with male sperm parameters and male fertility e.g. cancer, can be used to develop future strategies for male fertility preservation.

Hence, according to specific embodiments, the methods and cells described herein can enable future reproduction of prepubertal boys that undergo aggressive chemo/radio therapy from their own germ line, as further described infra.

According to specific embodiments, the methods and cells described herein can enable future reproduction of a subject having a disease associated with male sperm parameters and male fertility e.g. cancer from its own germ line, as further described infra.

According to specific embodiments, the methods of maturating human spermatogonium in vitro as described herein can be used along with the advanced intracytoplasmic sperm injection (ICSI) and in-vitro fertilization techniques to facilitate fertilization, as further described infra.

Thus, according to an aspect of the present invention, there is provided a method of in vitro proliferation and/or maturation of spermatogonium, comprising culturing said spermatogonium in a three-dimensional methylcellulose culture system (MCS) in a culture medium which comprises an effective concentration of a factor selected from the group consisting of granulocyte macrophages-colony stimulating factor (GM-CSF), interleukin-1 alpha (IL-1α), interleukin-1 beta (IL-1β) and interleukin-6 (IL-6), under conditions capable of inducing proliferation of said spermatogonium and/or differentiating said spermatogonium into at least meiotic and/or postmeiotic cells, thereby in vitro proliferating and/or maturing the spermatogonium.

According to an additional or an alternative aspect of the present invention, there is provided a method of in vitro proliferation and/or maturation of spermatogonium, comprising culturing said spermatogonium in a three-dimensional methylcellulose culture system (MCS) in a culture medium which comprises an effective concentration of a granulocyte macrophages-colony stimulating factor (GM-CSF) and/or an effective concentration of interleukin-1 alpha (IL-1α) under conditions capable of inducing proliferation of said spermatogonium and/or differentiating said spermatogonium into at least meiotic and/or postmeiotic cells, thereby in vitro proliferating and/or maturing the spermatogonium.

According to an additional or an alternative aspect of the present invention, there is provided a method of in vitro proliferation and/or maturation of spermatogonium, comprising culturing said spermatogonium in a three-dimensional methylcellulose culture system (MCS) in a culture medium which comprises Sertoli cells conditioned medium under conditions capable of inducing proliferation of said spermatogonium and/or differentiating said spermatogonium into at least meiotic and/or postmeiotic cells, thereby in vitro proliferating and/or maturing the spermatogonium.

As used herein the term “spermatogonium” refers to an undifferentiated male germ cell with a self-renewing capacity representing the first stage of spermatogenesis.

According to specific embodiments, the spermatogonium if a human spermatogonium.

Spermatogonia undergo spermatogenesis to form mature spermatozoa in the seminiferous tubules of the testis. There are three major subtypes of spermatogonia in humans:

(i) Type A (dark) cells, with dark nuclei. These cells are reserve spermatogonial stem cells which do not usually undergo active mitosis;

(ii) Type A (pale) cells, with pale nuclei. These are the spermatogonial stem cells that undergo active mitosis. These cells divide to produce Type B cell;

(iii) Type B cells, which divide to give rise to primary spermatocytes.

As used herein, the term “proliferation” refers to an increase in the number of cells in a population by means of cell division. Methods of evaluating proliferation are well known in the art and include, but not limited to, proliferation assays such as CFSE and BrDU and determining cell number by direct cell counting and microscopic evaluation.

As used herein the term “maturation” refers to the differentiation of a premeiotic spermatogonium into at least the meiotic, the postmeiotic stage and/or the mature elongated sperm.

According to specific embodiments, the maturation process results in meiotic and/or postmeiotic cells.

It should be noted that each of the stages in spermatogonium maturation is characterized by typical morphological and/or molecular markers, such as cell surface expression markers, which can be used to evaluate maturation by methods well known in the art such as microscopic evaluation, northern blow, western blot, immunostaining, flow cytometry, as further described hereinbelow.

The premeiotic spermatogonium stage includes several cells, such as type A cell which appears as a single cell (termed “As”), a type A cell that appears as a pair of two identical type A cells (termed “Apr”), a type A cell that appears as aligned cells (termed “Aal”), type A cells that are more differentiated (termed “A1-4”), intermediate stage cells (termed “In”), and a type B cell. Markers characteristics of As, Apr, Aal cells include, but are not limited to PLZF (promyelocytic leukaemia zinc finger), GFR-alpha1 (GDNF family receptor alpha-1), SALL4 (spalt like transcription factor 4), OCT4 (octamer-binding transcription factor 4), CD9 (CD9 molecule), alpha-6 integrin, and VASA [also known as “DEAD-box helicase 4 (DDX4)” or “MVH”). The expression of the marker C-kit (proto-oncogene receptor tyrosine kinase) is also characteristics of the most differentiating premeiotic cells. Markers characteristics of A1-4, In and B cells include but are not limited to CD9, alpha-6 integrin, VASA, and C-kit.

The meiotic spermatogonium includes several typical cells, such as type spermatocyte (termed “SPC”) and round spermatid (termed “RS”). Markers characteristics of SPC include alpha-6 integrin, VASA, c-kit, LDH (Lactate Dehydrogenase), BOULE (boule homolog, RNA binding protein), CREM (cAMP responsive element modulator), and ACR (acrosin). Markers characteristics of RS include, but are not limited to VASA, c-KIT, LDH, BOULE, CREM, and ACR.

The postmeiotic spermatogonium includes the RS and elongated sperm (termed “ES”) cells. Markers characteristics of the RS cells include, but are not limited to VASA, LDH, BOULE, CREM, PROT (protamine), and ACR (acrosin). Markers characteristics of the ES cells include, but are not limited to, the PROT and ACR markers.

A mature elongated sperm can be characterized by expression of at least one marker of the following cell surface markers: ACR (acrosin) and protamine. Morphological features of a meiotic spermatogonium include, for example, changes in the shape of the nucleus, and size of the cells (small).

According to some embodiments of the invention, the in vitro method of some embodiments of the invention results in maturation of the spermatogonium into a cell which expresses at least one of ACR (acrosin), CREM and BOULE.

According to some embodiments of the invention, the cell resulting from the method of some embodiments of the invention is characterized by the expression of at least one of ACR (acrosin), CREM and BOULE.

According to some embodiments of the invention, the in vitro method of some embodiments of the invention results in maturation of the spermatogonium into a cell which expresses ACR (acrosin) (Gene ID: 49).

According to some embodiments of the invention, the cell resulting from the method of some embodiments of the invention is characterized by the expression of ACR (acrosin).

According to some embodiments of the invention, the in vitro method of some embodiments of the invention results in maturation of the spermatogonium into a cell which expresses CREM.

According to some embodiments of the invention, the cell resulting from the method of some embodiments of the invention is characterized by the expression of CREM.

It should be noted that “CREM” (Gene ID: 1390), a cAMP responsive element modulator, is a bZIP transcription factor that binds to the cAMP responsive element found in many viral and cellular promoters. It is an important component of cAMP-mediated signal transduction during the spermatogenetic cycle, as well as other complex processes.

According to some embodiments of the invention, the in vitro method of some embodiments of the invention results in maturation of the spermatogonium into a cell which expresses BOULE (Gene ID: 66037).

According to some embodiments of the invention, the cell resulting from the method of some embodiments of the invention is characterized by the expression of BOULE.

According to some embodiments of the invention, the spermatogonium is an isolated spermatogonium.

According to some embodiments of the invention, the human spermatogonium is an isolated human spermatogonium.

The term “isolated” refers to at least partially separated from the natural environment e.g., from a subject (e.g., human).

Hence, according to specific embodiments, the method comprising obtaining the spermatogonium from a subject prior to the in vitro culturing.

Methods of isolating a spermatogonium are well known in the art. Thus, for example, the spermatogonium (e.g. human spermatogonium) can be isolated from at least part of a testis tissue of a subject.

According to some embodiments of the invention, the spermatogonium (e.g. human spermatogonium) is comprised in a testicular biopsy of the subject. According to some embodiments of the invention, the testicular biopsy is obtained from a prepubertal male subject.

According to some embodiments of the invention, the testicular biopsy is obtained from a non-obstructive azoospermic patient.

According to some embodiments of the invention, the testicular biopsy is a fresh tissue biopsy (removed from the testis of the subject).

According to some embodiments of the invention, the testicular biopsy is a frozen tissue biopsy, e.g., obtained by cryopreservation, e.g., as is further described hereinunder.

According to some embodiments of the invention, when using a frozen testicular biopsy, the testicular tissue biopsy is thawed to room temperature prior to culturing in the methylcellulose culture system of some embodiments of the invention.

Cryopreservation of testicular cells can be done by contacting a testicular tissue biopsy with a cryoprotectant (e.g., for 10 minutes in room temperature) and thereafter storing the tissue biopsy in liquid nitrogen for several months or years.

Briefly, for cryopreservation, after washing with a buffer such as PBS to remove residual blood, the biopsy is divided into small pieces, e.g., of about 3 mm³ each, and cryopreserved in 1.8 cryovials that contain 1.5 ml cryoprotectant media which can contain Dimethyl sulfoxide (DMSO) (e.g., about 5%), albumin (e.g., 10% human serum albumin) and sucrose (e.g., about 3.5% diluted in Hanks' Balanced Salt solution (HBSS)). The cooling rate of the biopsy can be gradual from about 37° C., through about 0° C. to about −80° C. For example, the cooling rate can be 0.5° C./minute, with holding at 0° C. for 9 minutes, followed by a cooling rate of 0.5° C./minute, until −8° C. with a holding of 5 minutes at this temperature. After 15 minutes holding at −8° C., the vials can be frozen to −40° C. at a rate of 0.5° C./min. The vials can then be frozen to −80° C. at a rate of 0.7° C./min and then transferred to liquid nitrogen. The cryopreserved biopsy can be thawed in room temperature (RT) and centrifuged (for washing) in the presence of any suitable culture medium such as Minimum Essential Media (MEM) (Biological Industries).

As used herein, the term “culturing” refers to at least spermatogonium and culture medium in an in vitro environment. The culture is maintained under conditions capable of inducing proliferation of the spermatogonium and/or differentiating the spermatogonium into at least meiotic and/or postmeiotic cells. Such conditions include for example an appropriate temperature (e.g., 37° C.), atmosphere (e.g., air plus 5% CO₂), pH, light, medium, supplements and the like.

The culture may be in a glass, plastic or metal vessel that can provide an aseptic environment for cell culturing. According to specific embodiments, the culture vessel includes dishes, plates, flasks, bottles, vials, bags, bioreactors or any device that can be used to grow cells.

According to the method of some embodiments of the invention, culturing the spermatogonium is performed in a three-dimensional methylcellulose culture system (MCS).

The methylcellulose is used as a three-dimensional matrix or scaffold, to support the growth and/or differentiation (or maturation) of the spermatogonium.

As used herein the term “scaffold” or “matrix”, which are interchangeably used herein, refers to a two-dimensional or a three-dimensional supporting framework.

According to some embodiments of the invention, the scaffold is a three-dimensional scaffold.

According to some embodiments of the invention, the scaffold enables the proliferation and/or differentiation of the spermatogonium into at least the meiotic and/or postmeiotic stage and/or a mature sperm.

According to some embodiments of the invention, the scaffold is a methylcellulose scaffold.

Methylcellulose is a synthetic (non-natural) chemical compound derived from cellulose. It is available as a hydrophilic white powder, preferably in a pure form which can be dissolved in cold water, forming a clear viscous solution or gel, or in an already dissolved ready to use viscous solution. Methylcellulose is synthetically produced by heating cellulose with caustic solution (e.g. a solution of sodium hydroxide) and treating it with methyl chloride. In the substitution reaction that follows, the hydroxyl residues (—OH functional groups) are replaced by methoxide (—OCH3 groups).

Different kinds of methylcellulose can be prepared depending on the number of hydroxyl groups substituted. Cellulose is a polymer consisting of numerous linked glucose molecules, each of which exposes three hydroxyl groups. The Degree of Substitution (DS) of a given form of methylcellulose is defined as the average number of substituted hydroxyl groups per glucose. The theoretical maximum is thus a DS of 3.0, however more typical values are 1.3-2.6. Different methylcellulose preparations can also differ in the average length of their polymer backbones.

The methylcellulose can be obtained from various suppliers such as R&D Systems™, Minneapolis, USA, and Sigma-Aldrich®.

Suitable concentrations of methylcellulose matrixes include from about 35% to about 50% (volume/volume), e.g., between 40-50% (v/v), e.g., between 40-45% (v/v), e.g., at a concentration of about 42% (v/v).

According to some embodiments of the invention, the cells are directly added into a culture system which includes a suitable culture medium and a methylcellulose matrix.

Additionally or alternatively, the cells which are seeded onto the methylcellulose culture system (the MC matrix and culture medium) are mainly non-adherent cells. It should be noted that the present inventor has uncovered that seeding of adherent cells in the MCS is less efficient than seeding of isolated cells which are mainly (e.g., more than 50%, more than 60%, more than 70%, more than 80%, more than 90%) non-adherent cells.

According to some embodiments of the invention, prior to culturing in the methylcellulose culture systems (MCS) the cells are cultured in a culture medium under conditions which enable removal of adherent cells, while isolating at least 50%, at least 60%, at least 70%, at least 80%, at least 90%, at least 95% or more of the non-adherent cells. For example, according to specific embodiments, the cells of the testis tissue biopsy are cultured in a tissue culture plate, such as a multi-well plate, e.g., a 24-well plate, preferably in uncoated wells of a tissue-culture grade flask. The cells are seeded at a concentration of about 4-5×10⁴ cells/well/500 μl, e.g., at about 2×10⁴ cells/well/500 μl in a culture medium (base medium) such as MEM medium which improves adherence of adherent cells to the plastic. A suitable basic culture medium may comprise sodium bicarbonate (e.g., at a concentration of 7.5%), L-glutamine (e.g., at a concentration of 200 mM), non-essential amino acids (e.g., at a concentration of 1%), penicillin/streptomycin and gentamicin (e.g., at a concentration of 10 mg/ml), and incubated for about 2 nights at 37° C., 5% CO₂.

The non-adherent cells are then collected and cultured (at a concentration of about 2-5×10⁴ cells/well/500 μl; or at a concentration of 4-5×10⁴ cells/well/500 μl) in methylcellulose as a three-dimensional (3D) culture system.

The cells are usually diluted in a culture medium prior to the addition of the culture medium onto the methylcellulose scaffold. For example, if 42% of methylcellulose is used, then the cells are diluted in the remaining 58% of culture medium. However, it is appreciated that the cells can be also added to an already mixed medium and methylcellulose culture system.

The culture medium used in the MC culture system can include a base medium supplemented with serum and/or serum replacement, as described hereinunder. For example, in the case of using 42% methylcellulose matrix, the cells can be diluted in the remaining 58% of culture medium which can comprise 33% StemPro-34 medium and 25% KSR (knock-out serum replacement) (Gibco, USA) enriched with different factors, ingredients and reagents described herein below.

Alternatively or additionally, the cells can be diluted in a medium which contains 33% StemPro-34 medium (Gibco, e.g., from USA) and the StemPro supplement (e.g., at a concentration of 2.6%; e.g., from Gibco), and optionally with the addition of insulin (e.g., at a concentration of 25 μg/ml; e.g., from Gibco), transferrin (e.g., at a concentration of 100 μg/ml; e.g., from Gibco), putrescin (e.g., at a concentration of 60 μg/ml; Gibco), sodium selenite (e.g., at a concentration of 30 nM; e.g., from Gibco), D-glucose (e.g., at a concentration of 6 mg/ml; e.g., from Sigma), pyruvic acid (e.g., at a concentration of 30 μg/ml; e.g., from Sigma), bovine serum albumin (BSA) (e.g., at a concentration of 5 mg/ml; Millpore, Illkirch, France), L-glutamine (e.g., at a concentration of 2 mM; e.g., from Biological Industries), 2-mercaptoethanol (e.g., at a concentration of 0.5 μM; e.g., from Gibco), MEM vitamin solution (e.g., at a concentration of 10 μl/ml; e.g., from Gibco, UK), MEM non-essential amino acid solution (e.g., at a concentration of 10 μl/ml; e.g., from Gibco, UK), ascorbic acid (e.g., at a concentration of 100 μM; e.g., from Sigma, China), d-biotin (e.g., at a concentration of 10 μg/ml; e.g., from Sigma), KSR (e.g., at a concentration of 1%, e.g., from Gibco, UK), Pen/Strep (e.g., from Biological Industries), enriched with different factors as described hereinunder.

According to specific embodiments, for establishment of the methylcellulose (MC) culture system, a culture medium containing the isolated cells from the testicular biopsy (e.g., 58% final dilution in the well) are mixed with MC (e.g., 42% final dilution in the well) and are cultured in the wells.

According to specific embodiments, cells were cultured for 1-16 weeks, e.g., for about 1-15, 2-15, 5-15, 1-10, 1-5 or 2-5 weeks in CO₂ incubator at 37° C.

Every 7-14 days a fresh concentrated medium (×10) can be added, e.g., 50 μl/well of fresh concentrated (×10) enriched StemPro-34 medium [containing all the factors (e.g. growth factors) used in the primary culture] to the cell cultures to be followed up after additional 1-2 weeks.

As described, the culture medium used for culturing the spermatogonium comprises various factors e.g., growth factors, cytokines and/or hormones.

According to specific embodiments, the culture medium comprises a factor selected from the group consisting of granulocyte macrophages-colony stimulating factor (GM-CSF), interleukin-1 alpha (IL-1α), interleukin-1 beta (IL-1β) and interleukin-6 (IL-6).

According to specific embodiments, the culture medium comprises GM-CSF.

“Granulocyte Macrophage Colony Stimulating Factor (GM-CSF)” (Gene ID: 1437), also known as CSF2, molgramostin and sargramostin. According to specific embodiments, the GM-CSF protein refers to the human protein, such as provided in the following GenBank Number NP_000749 (SEQ ID NO: 27).

Any of the proteinaceous factors described herein also encompass functional homologues (naturally occurring or synthetically/recombinantly produced), orthologs (from other species) which exhibit the desired activity (i.e., inducing in-vitro proliferation and/or maturation of spermatogonium under the culture conditions described herein). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide described herein or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

The functional homologs also refer to functional portions which maintain the activity of the full length protein as defined herein.

Sequence identity or homology can be determined using any protein or nucleic acid sequence alignment algorithm such as Blast, ClustalW, and MUSCLE.

According to specific embodiments, the GM-CSF is a recombinant GM-CSF.

GM-CSF can be commercially obtained from e.g. R&D Systems™, Sigma-Aldrich®, ThermoFisher Scientific.

According to specific embodiments, the level of GM-CSF in the cultures and methods of the present invention is above the level obtained in a spermatogonium culture of the same origin under the same culture conditions without the addition of GM-CSF.

According to some embodiments of the invention, the effective concentration of GM-CSF is in the range of 0.01-100 ng/ml, 0.1-100 ng/ml, 0.01-10 ng/ml, 0.1-10 ng/ml, 0.1-1 ng/ml.

According to specific embodiments, the culture medium comprises IL-1α.

“Interleukin-1 alpha (IL-1α)” (Gene ID: 3552), also known as hematopoietin 1. According to specific embodiments, the IL-1a protein refers to the human protein, such as provided in the following GenBank Number NP_000566 or NP_001358483 (SEQ ID NO: 28-29).

According to specific embodiments, the IL-1α is a recombinant IL-1α.

IL-1α can be commercially obtained from e.g. R&D Systems™, Sigma-Aldrich®, Biolegend®.

According to specific embodiments, the level of IL-1α in the cultures and methods of the present invention is above the level obtained in a spermatogonium culture of the same origin under the same culture conditions without the addition of IL-1α.

According to some embodiments of the invention, the effective concentration of IL-1α is in the range of 0.1-1000 pg/ml, 0.1-100 pg/ml, 0.1-10 pg/ml, 1-10 pg/ml, e.g., about 10 pg/ml.

According to specific embodiments, the culture medium comprises IL-1β.

“Interleukin-1 beta (IL-1β)” (Gene ID: 3553), also known as leukocytic pyrogen, leukocytic endogenous mediator, mononuclear cell factor, lymphocyte activating factor. According to specific embodiments, the IL-1β protein refers to the human protein, such as provided in the following GenBank Number NP_000567 (SEQ ID NO: 30).

According to specific embodiments, the IL-1β is a recombinant IL-1β.

IL-1β can be commercially obtained from e.g. R&D Systems™, Sigma-Aldrich®, Biolegend®.

According to specific embodiments, the level of IL-1β in the cultures and methods of the present invention is above the level obtained in a spermatogonium culture of the same origin under the same culture conditions without the addition of IL-1β.

According to some embodiments of the invention, the effective concentration of IL-1β is in the range of 0.1-1000 pg/ml, 0.1-100 pg/ml, 1-100 pg/ml.

According to specific embodiments, the culture medium comprises IL-6 (Gene ID 3569.

According to specific embodiments, the IL-6 protein refers to the human protein, such as provided in the following GenBank Number NP_000591, NP_001305024 or NP_001358025 (SEQ ID NO: 31-33).

According to specific embodiments, the IL-6 is a recombinant IL-6.

IL-6 can be commercially obtained from e.g. R&D Systems™, Sigma-Aldrich®, Biolegend®.

According to specific embodiments, the level of IL-6 in the cultures and methods of the present invention is above the level obtained in a spermatogonium culture of the same origin under the same culture conditions without the addition of IL-6.

According to some embodiments of the invention, the effective concentration of IL-6 is in the range of 0.1-1000 pg/ml, 0.1-100 pg/ml, 1-100 pg/ml.

According to some embodiments of the invention, the conditions comprise culturing the spermatogonium (e.g. human spermatogonium) in a culture medium which comprises an effective concentration of at least one growth factor selected from the group consisting of Glial cell line-derived neurotrophic factor (GDNF), leukemia inhibitory factor (LIF), basic fibroblast growth factor (bFGF), and epidermal growth factor (EGF).

According to some embodiments of the invention, the culture medium further comprises an effective concentration of TNFalpha (TNFα).

According to some embodiments of the invention, the culture medium further comprises an effective concentration of at least one agent selected from the group consisting of: testosterone, and retinoic acid.

According to some embodiments of the invention, the method further comprising culturing the human spermatogonium in the presence of testosterone.

According to some embodiments of the invention, the conditions comprise culturing said human spermatogonium in a culture medium which comprises an effective concentration of at least one growth factor selected from the group consisting of TNFalpha (TNFα), Glial cell line-derived neurotrophic factor (GDNF), leukemia inhibitory factor (LIF) and basic fibroblast growth factor (bFGF).

According to some embodiments of the invention, the conditions comprise culturing the human spermatogonium in a culture medium which comprises an effective concentration of at least one growth factor selected from the group consisting of GDNF, LIF, and bFGF.

According to some embodiments of the invention, the conditions comprise culturing the human spermatogonium in a culture medium which comprises an effective concentration of at least one growth factor selected from the group consisting of GDNF, LIF, and EGF.

According to some embodiments of the invention, the conditions comprise culturing the human spermatogonium in a culture medium which comprises an effective concentration of at least one growth factor selected from the group consisting of GDNF, bFGF, and EGF.

According to some embodiments of the invention, the conditions comprise culturing the human spermatogonium in a culture medium which comprises an effective concentration of at least one growth factor selected from the group consisting of LIF, bFGF, and EGF.

According to some embodiments of the invention, the conditions comprise culturing said human spermatogonium in a culture medium which comprises an effective concentration of Glial cell line-derived neurotrophic factor (GDNF).

Glial cell line-derived neurotrophic factor (GDNF) (Gene ID 2668) is a secreted ligand of the TGF-beta (transforming growth factor-beta) superfamily of proteins. Ligands of this family bind various TGF-beta receptors leading to recruitment and activation of SMAD family transcription factors that regulate gene expression.

According to specific embodiments, the GDNF protein refers to the human protein, such as provided in the following GenBank Number NP_000505, NP_001177397, NP_001177398, NP_001265027 or NP_954701 (SEQ ID NO: 34-38).

According to specific embodiments, the GDNF is a recombinant GDNF.

GDNF can be provided from various suppliers and makers, such as from Biolegend® (CA, USA), PROSPEC Protein Specialists (Rehovot, Ill.), and ThermoFisher Scientific.

According to some embodiments of the invention, the effective concentration of GDNF is in the range of 1-50 ng/ml, e.g., 1-40 ng/ml, e.g., 1-30 ng/ml, e.g., 1-25 ng/ml, e.g., 1-20 ng/ml, e.g., about 5 ng/ml, e.g., about 10 ng/ml, e.g., about 15 ng/ml, e.g., about 20 ng/ml.

According to some embodiments of the invention, the conditions comprise culturing said human spermatogonium in a culture medium which comprises an effective concentration of leukemia inhibitory factor (LIF).

Leukemia inhibitory factor (LIF) (Gene ID 3976) is a pleiotropic cytokine with roles in several different systems. It is involved in the induction of hematopoietic differentiation in normal and myeloid leukemia cells, induction of neuronal cell differentiation, regulator of mesenchymal to epithelial conversion during kidney development, and may also have a role in immune tolerance at the maternal-fetal interface.

According to specific embodiments, the LIF protein refers to the human protein, such as provided in the following GenBank Number NP_001244064 or NP_002300 (SEQ ID NO: 39-40).

According to specific embodiments, the LIF is a recombinant LIF.

LIF can be provided from various suppliers and makers, such as from Biolegend® (CA, USA), PEPROTECH® (Rehovot, Ill.), and Sigma-Aldrich® (MERCK).

According to some embodiments of the invention, the effective concentration of LIF is in the range of 1-50 ng/ml, e.g., 1-40 ng/ml, e.g., 1-30 ng/ml, e.g., 1-25 ng/ml, e.g., 1-20 ng/ml, e.g., about 5 ng/ml, e.g., about 10 ng/ml, e.g., about 15 ng/ml, e.g., about 20 ng/ml.

According to some embodiments of the invention, the conditions comprise culturing said human spermatogonium in a culture medium which comprises an effective concentration of basic fibroblast growth factor (bFGF).

Basic fibroblast growth factor (bFGF) (Gene ID 2247) is a member of the fibroblast growth factor (FGF) family. FGF family members bind heparin and possess broad mitogenic and angiogenic activities. This protein has been implicated in diverse biological processes, such as limb and nervous system development, wound healing, and tumor growth.

According to specific embodiments, the bFGF protein refers to the human protein, such as provided in the following GenBank Number NP_001997 or NP_001348594 (SEQ ID NO: 41-42).

According to specific embodiments, the bFGF is a recombinant bFGF.

bFGF can be provided from various suppliers and makers, such as from Biolegend® (CA, USA), and Invitrogen Corporation products (Grand Island N.Y., USA).

According to some embodiments of the invention, the effective concentration of bFGF is in the range of 1-50 ng/ml, e.g., 1-40 ng/ml, e.g., 1-30 ng/ml, e.g., 1-25 ng/ml, e.g., 1-20 ng/ml, e.g., about 5 ng/ml, e.g., about 10 ng/ml, e.g., about 15 ng/ml, e.g., about 20 ng/ml.

According to some embodiments of the invention, the conditions comprise culturing said human spermatogonium in a culture medium which comprises an effective concentration of epidermal growth factor (EGF).

Epidermal growth factor (EGF) (Gene ID 1950) is a member of the epidermal growth factor superfamily. EGF acts a potent mitogenic factor that plays an important role in the growth, proliferation and differentiation of numerous cell types. This protein acts by binding with high affinity to the cell surface receptor, epidermal growth factor receptor. Defects in the EGF gene are the cause of hypomagnesemia type 4, and dysregulation of the EGF gene has been associated with the growth and progression of certain cancers. Alternative splicing results in multiple transcript variants, at least one of which encodes a preproprotein that is proteolytically processed.

According to specific embodiments, the EGF protein refers to the human protein, such as provided in the following GenBank Number NP_001171601, NP_001171602, NP_001954 or NP_001343950 (SEQ ID NO: 43-46).

According to specific embodiments, the EGF is a recombinant EGF.

EGF can be provided from various suppliers and makers, such as from Biolegend® (CA, USA), ALMONE LABS (Hadassah Ein Kerem, Ill.), PROSPEC Protein Specialists (Rehovot, Ill.), and ACRO BIOSYSTEMS (St. Louis, Mo., USA). According to some embodiments of the invention, the effective concentration of EGF is in the range of 0.1-200 ng/ml, e.g., 0.5-100 ng/ml, e.g., 1-80 ng/ml, e.g., 1-50 ng/ml, e.g., 5-50 ng/ml, e.g., about 5-25 ng/ml, e.g., between 18-25 ng/ml, e.g., between 19-21 ng/ml, e.g., about 5 ng/ml, 10 ng/ml, e.g., about 15 ng/ml, e.g., about 20 ng/ml, e.g., about 20 ng/ml (e.g., 20 ng/ml).

According to some embodiments of the invention, the conditions comprise culturing said human spermatogonium in a culture medium which comprises an effective concentration of TNFalpha (TNFα).

Tumor necrosis factor (TNF) alpha (TNFα) (Gene ID 7124) is a multifunctional proinflammatory cytokine that belongs to the tumor necrosis factor (TNF) superfamily. This cytokine is mainly secreted by macrophages and can bind to the TNFRSF1A/TNFR1 and TNFRSF1B/TNFBR receptors.

According to specific embodiments, the TNFα protein refers to the human protein, such as provided in the following GenBank Number NP_000585 (SEQ ID NO: 47).

According to specific embodiments, the TNFα is a recombinant TNFα.

TNFα can be provided from various suppliers and makers, such as from ALMONE LABS (Hadassah Ein Kerem, Ill.), PROSPEC Protein Specialists (Rehovot, Ill.), and Biolegend®, CA, USA.

According to some embodiments of the invention, the effective concentration of TNFalpha (TNFα) is in the range of 1-200 pg/ml, e.g., 1-100 pg/ml, e.g., 1-50 pg/ml, e.g., about 5 pg/ml, e.g., about 10 pg/ml, e.g., about 15 pg/ml, e.g., about 20 pg/ml, e.g., about 25 pg/ml, e.g., about 30 pg/ml, e.g., about 35 pg/ml, e.g., about 40 pg/ml, e.g., about 50 pg/ml.

According to some embodiments of the invention, the culture medium is devoid of TNFalpha (TNFα).

According to specific embodiments, the medium comprises testosterone.

Testosterone is the primary male sex hormone and an anabolic steroid from the androstane class containing a keto and hydroxyl groups at the three and seventeen positions respectively. It is biosynthesized in several steps from cholesterol and is converted in the liver to inactive metabolites. It exerts its action through binding to and activation of the androgen receptor. In male humans, testosterone plays a key role in the development of male reproductive tissues such as testes and prostate, as well as promoting secondary sexual characteristics. Testosterone can be obtained from various sources and suppliers such as Sigma-Aldrich® (MERCK), Bayer.

According to some embodiments of the invention, the effective concentration of testosterone is in the range of 1×10⁻⁸ M (molar) through 1×10⁻⁶ M, e.g., 1×10⁻⁸ M, e.g., 1×10⁻⁷ M, e.g., 1×10⁻⁶ M.

According to specific embodiments, the culture medium comprises Follicle stimulating hormone (FSH).

Follicle stimulating hormone (FSH) is a gonadotropin, a glycoprotein polypeptide hormone, which is synthesized and secreted by the gonadotropic cells of the anterior pituitary gland, and regulates the development, growth, pubertal maturation, and reproductive processes of the body.

FSH can be obtained from various sources and suppliers such as Sigma-Aldrich® (MERCK), Serono.

According to specific embodiments, the culture medium is devoid of FSH.

According to specific embodiments, the culture medium is devoid of a combination of testosterone and FSH and/or a combination of testosterone and TNFα.

According to specific embodiments, the culture medium comprises retinoic acid.

Retinoic acid is a metabolite of vitamin A (retinol) that mediates the functions of vitamin A required for growth and development. During early embryonic development, retinoic acid generated in a specific region of the embryo helps determine position along the embryonic anterior/posterior axis by serving as an intercellular signaling molecule that guides development of the posterior portion of the embryo.

Retinoic acid can be obtained from various sources and suppliers such as Sigma-Aldrich® (MERCK).

According to some embodiments of the invention, the effective concentration of retinoic acid is in the range of 1×10⁻⁸ M (molar) through 1×10⁻⁶ M, e.g., 1×10⁻⁸ M, e.g., 1×10⁻⁷ M, e.g., 1×10⁻⁶ M.

According to some embodiments of the invention, the at least one hormone (e.g. testosterone) is added in the beginning of the culturing process, along with the at least one factor (e.g. growth factor, cytokine) described hereinabove.

Thus, according to specific embodiments, the hormone (e.g. testosterone) is added to the culture following a pre-determined time period.

Hence, according to specific embodiments, the method further comprises culturing cells developed following a pre-determined time period from said spermatogonium in a second culture medium which comprises the factor disclosed herein and an effective concentration of testosterone.

Determining the time period for addition of the hormone (e.g. testosterone) is well within the capabilities of the skilled in the art. According to a specific embodiment, the predetermined time period is 1-8 weeks, 1-5 weeks, 2-4 weeks or two weeks±2 days.

According to some embodiments of the invention, the culturing in the presence of the at least one hormone is performed following about one month of culturing in the presence of the at least one factor (e.g. growth factor, cytokine) described herein, e.g., following about two months of culturing in the presence of said at least one factor (e.g. growth factor, cytokine) described herein.

According to specific embodiments, the second culture medium comprises all factors and substances comprised in the first culture medium.

According to some embodiments of the invention, the at least one hormone is added to the culture medium which comprises the at least one factor (e.g. growth factor, cytokine).

According to some embodiments of the invention, the at least one hormone is added to a culture medium which comprises at least one factor selected from the group consisting of GM-CSF, IL-1α, IL-1β and IL-6.

According to some embodiments of the invention, the at least one hormone is added to a culture medium which comprises GM-CSF and/or IL-1α.

According to some embodiments of the invention, the at least one hormone is added to a culture medium which comprises at least one growth factor from the growth factors selected from the group consisting of: GDNF, LIF, bFGF, and EGF.

According to some embodiments of the invention, the at least one hormone is added to a culture medium which comprises at least 2 growth factors (e.g., at least 2 factors from the growth factors selected from the group consisting of: GDNF, LIF, bFGF, and EGF).

According to some embodiments of the invention, the at least one hormone is added to a culture medium which comprises at least 3 growth factors (e.g., at least 3 growth factors from the growth factors selected from the group consisting of: GDNF, LIF, bFGF, and EGF).

According to some embodiments of the invention, the at least one hormone is added to a culture medium which comprises at least 4 growth factors (e.g., at least GDNF, LIF, bFGF, and EGF).

Following addition of the medium comprising the hormone (e.g. testosterone) the culturing is effected for a second predetermined time period. Determining the second culturing time period is well within the capabilities of the skilled in the art. According to a specific embodiment, the predetermined time period is 1-10 weeks, 1-5 weeks, 2-4 weeks or two weeks±2 days.

As used herein the phrase “culture medium” refers to a liquid substance used to support the growth of spermatogonial cells and optionally induce their proliferation and/or differentiation to meiotic and postmeiotic stages including the generation of sperm-like cells in 3D in vitro culture of MCS. The culture medium used according to some embodiments of the invention can be a water-based medium which includes a combination of substances such as salts, nutrients, minerals, vitamins, amino acids, nucleic acids, and/or proteins such as cytokines, growth factors and hormones, all of which are needed for cell proliferation and/or differentiation of spermatogonial cells into meiotic and postmeiotic stages including the generation of sperm-like cells in 3D in vitro culture of MCS.

For example, a culture medium according to an aspect of some embodiments of the invention can be a synthetic tissue culture medium such as the StemPro® (Thermo Fisher Scientific), Ko-DMEM (Gibco-Invitrogen Corporation products, Grand Island, N.Y., USA), DMEM/F12 (Biological Industries, Biet HaEmek, Israel), RPMI (Biological Industries, Biet HaEmek, Israel), supplemented with the necessary additives as is further described herein.

According to some embodiments of the invention, the culture medium comprises serum replacement.

Serum replacement is usually added to most culture media which are designed for culturing stem cells or progenitor cells, in order to provide the cells with the optimal environment, similar to that present in vivo (i.e., within the organism from which the cells are derived, e.g., for a developing spermatogonium in the testis).

Serum replacement is used in culture media to replace the need of a serum. While the use of serum which is derived from either an animal source (e.g., bovine serum) or a human source (human serum) is limited by the significant variations in serum components between individuals and the risk of having xeno contaminants (in case of an animal serum is used), the use of the more defined composition such as the currently available serum Replacement™ (Gibco-Invitrogen Corporation, Grand Island, N.Y. USA) may be limited by the presence of Albumax (Bovine serum albumin enriched with lipids) which is from an animal source within the composition (International Patent Publication No. WO 98/30679 to Price, P. J. et al).

Various animal-free formulations of serum replacement are available for in vitro culturing.

According to some embodiments of the invention, the culture medium comprises STEM PRO® (Thermo Fisher Scientific) supplement.

The StemPro® (Thermo Fisher Scientific) hESC SFM (serum free medium) is a fully-defined serum- and feeder-free medium specifically formulated for the growth and expansion of human embryonic stem cells (hESCs). The StemPro® (Thermo Fisher Scientific) hESC SFM includes DMEM-12 with GlutaMAX™ (Thermo Fisher Scientific) medium, StemPro® hESC Supplement and Bovine serum albumin 25% (BSA). The Gibco™ GlutaMAX™ media contains a stabilized form of L-glutamine, L-alanyl-L-glutamine, preventing degradation and ammonia build-up even during long-term cultures.

It should be noted that when an animal-contaminant-free serum replacement is used to culture human cells, then the serum replacement is referred to as being “xeno-free”.

The term “xeno” is a prefix based on the Greek word “Xenos”, i.e., a stranger. As used herein the phrase “xeno-free” refers to being devoid of any components/contaminants which are derived from a xenos (i.e., not the same, a foreigner) species.

For example, a xeno-free serum replacement for use with human cells (i.e., an animal contaminant-free serum replacement) can include a combination of insulin, transferrin and selenium. Additionally or alternatively, a xeno-free serum replacement can include human or recombinantly produced albumin, transferrin and insulin.

Non-limiting examples of commercially available xeno-free serum replacement compositions include the premix of ITS (Insulin, Transferrin and Selenium) available from Invitrogen corporation (ITS, Invitrogen, Catalogue No. 51500-056); Serum replacement 3 (SR3; Sigma, Catalogue No. S2640) which includes human serum albumin, human transferring and human recombinant insulin and does not contain growth factors, steroid hormones, glucocorticoids, cell adhesion factors, detectable Ig and mitogens; KnockOut™ SR XenoFree [Catalogue numbers A10992-01, A10992-02, part Nos. 12618-012 or 12618-013, Invitrogen GIBCO] which contains only human-derived or human recombinant proteins.

According to some embodiments of the invention, the ITS (Invitrogen corporation) or SR3 (Sigma) xeno-free serum replacement formulations are diluted in a 1 to 100 ratio in order to reach a ×1 working concentration.

According to some embodiments of the invention, the concentration of the serum replacement [e.g., KnockOut™ SR XenoFree (Invitrogen)] in the culture medium is in the range of from about 1% [volume/volume (v/v)] to about 50% (v/v), e.g., from about 5% (v/v) to about 40% (v/v), e.g., from about 5% (v/v) to about 30% (v/v), e.g., from about 10% (v/v) to about 30% (v/v), e.g., from about 10% (v/v) to about 25% (v/v), e.g., from about 10% (v/v) to about 20% (v/v), e.g., about 10% (v/v), e.g., about 15% (v/v), e.g., about 20% (v/v), e.g., about 30% (v/v), e.g., about 25% (v/v).

Once in the MCS, the cells can be evaluated for their differentiation state. According to some embodiments of the invention, the cells in the MCS are evaluated every 10 days to 2 weeks under the microscope for the growth quality/viability and the morphology of the developed colonies.

It should be noted that about 10% of the cells may present apoptotic vacuoles in their cytoplasm.

According to some embodiments of the invention, the cells are cultured in the MC systems for about 3 months, in order to mimic the physiological timing of development of human spermatogenesis (around 3 months).

At the end of the incubation period in MCS, the cells can be collected by adding a buffer (e.g., PBS, e.g., an amount of 0.5 ml PBS to each culture well that contained 0.5 ml MC mix), further pipetting the buffer and collecting the suspension to a new tube (e.g., a 15 ml tube). The tubes are centrifuged (e.g., in 1600 RPM for 10 minutes) to remove the excess of culture medium and buffer. Most of the volume is removed and the remainder liquid (around 100 μl from the bottom of the tube) is collected. This volume which contains the differentiated cells, can be isolated and be further used for clinical purposes.

Additionally or alternatively, the differentiated cells can be smeared on a slide for further evaluations (e.g., histological evaluations), and/or can be collected and kept at −70° C. (to be used for RNA analyses). In case there are more than 10-15 small colonies, or 5 medium or large colonies in the well, the cells can be utilized for both IF and RNA analyses.

The fertilization capacity and epigenetics of the generated postmeiotic and sperm-like cells can be evaluated. Methods of qualifying the degree of meiotic differentiating are known in the art and include, for example, monitoring the expression levels of various premeiotic (e.g., VASA, SALL4, OCT4, PLZF, CD9, A-6-INTEGRIN, GFR-A1, and C-KIT), meiotic (e.g., CREM-1, LDH, ACROSIN, and BOULE) and postmeiotic (e.g., ACROSIN, and PROTAMINE) markers. Such methods include RNA or protein detection methods which are well known in the art. Non-limiting examples of such methods and exemplary antibodies and/or primers for RNA analysis are described hereinunder.

According to specific embodiments, the culture medium comprises a conditioned medium, such as a Sertoli cell conditioned medium.

Condition Medium

Conditioned medium is the growth medium of a monolayer cell culture (i.e., feeder cells) present following a certain culturing period. The conditioned medium includes growth factors and cytokines secreted by the monolayer cells in the culture.

Conditioned medium can be collected from a variety of cells forming monolayers in culture. Examples include, but are not limited to mouse Sertoli cells conditioned medium and human Sertoli cells conditions medium.

Such a growth medium can be any medium suitable for culturing feeder cells. The growth medium can be supplemented with nutritional factors, such as amino acids, (e.g., L-glutamine), anti-oxidants (e.g., beta-mercaptoethanol) and growth factors, which benefit the maturation of the spermatogonia cells. Serum or serum replacement can be added at effective concentration ranges.

Feeder cells are cultured in the growth medium for sufficient time to allow adequate accumulation of secreted factors to support maturation of the spermatogonia cells to meiotic and/or postmeiotic cells. Typically, the medium is conditioned by culturing for 4-24 hours at 37° C. However, the culturing period can be scaled by assessing the effect of the conditioned medium spermatogonia differentiation and maturation.

Selection of culture apparatus for conditioning the medium is based on the scale and purpose of the conditioned medium. Large-scale production preferably involves the use of dedicated devices. Continuous cell culture systems are reviewed in Furey (2000) Genetic Eng. News 20:10.

Following accumulation of adequate factors in the medium, growth medium (i.e., conditioned medium) is separated from the feeder cells and collected. It will be appreciated that the feeder cells can be used repeatedly to condition further batches of medium over additional culture periods, provided that the cells retain their ability to condition the medium.

Preferably, the conditioned medium is sterilized (e.g., filtration using a 20 μM filter) prior to use. The conditioned medium of some embodiments of the invention may be applied directly on the spermatogonia or extracted to concentrate the effective factor such as by salt filtration. For future use, conditioned medium is preferably stored frozen at −80° C.

According to specific embodiments, the conditioned medium comprises 10-40% (v/v) of the culture medium.

According to some embodiments of the invention, the method further comprises identifying and/or isolating a mature cell following the culturing. Methods of identifying and isolating such cells are well known in the art and include, for example, immunostaining and flow cytometry, as further described hereinabove and below.

As used herein, the term “mature cell” refers to a differentiated spermatogonial cell encompassing a cell at the meiotic, the postmeiotic stage and/or the mature elongated sperm.

According to specific embodiments, the mature cell is a meiotic and/or a postmeiotic cell.

Thus, according to specific embodiments, the method further comprises identifying and/or isolating s meiotic cell, a postmeiotic cell and/or a mature sperm cell resulting from the in vitro maturation.

According to some embodiments of the invention, the method further comprises identifying and/or isolating a meiotic cell and/or a postmeiotic cell resulting from the in vitro maturation.

According to some embodiments of the invention, identification and/or isolation of the meiotic cell, the postmeiotic cell and/or the mature sperm cell is by a characteristic marker to the meiotic cell, the postmeiotic cell and/or the mature sperm cell, respectively.

Thus, according to some embodiments of the invention, the method further comprises identifying and/or isolating a cells expressing at least one marker selected from the group consisting of ACR (acrosin), CREM and BOULE following the culturing.

According to some embodiments of the invention, the method further comprises identifying and/or isolating a cell expressing CREM resulting from the in vitro maturation of the spermatogonium.

According to some embodiments of the invention, the method further comprises identifying and/or isolating a cell expressing acrosin resulting from the in vitro maturation of the spermatogonium.

Methods of identifying a sperm cell (elongated cell) include, but are not limited to, using a mitochondrial staining (e.g., MitoTracker). A sperm cell has a concentrated mitochondria in the neck which intensely stains in green.

The advantage of using MitoTracker is to identify a few sperm cells that could be used for in vitro fertilization since the MitoTracker stains live cells without fixation.

According to an additional or an alternative aspect of the present invention, there is provided a cell obtainable according to the method.

According to specific embodiments, the cell obtainable by the method is characterized by at least the expression of ACR (acrosin), CREM and/or BOULE.

According to specific embodiments, the cell obtainable by the method is characterized by at least the expression of acrosin.

It should be appreciated that the methods described herein and the cells obtainable therefrom can be further used to facilitate fertilizing of an oocyte using e.g. in vitro fertilization (IVF) or advanced intracytoplasmic sperm injection (ICSI) or can be injected to a subject in need thereof (e.g. auto-transplantation).

That is, the methods described herein and the cells obtainable therefrom can be further used in a method of treating a subject in need thereof.

Hence, according to specific embodiments, the spermatogonium is obtained from a subject in need thereof.

As used herein, the term “subject” includes male mammals, preferably human beings at any age that have spermatogonial cells (SPGCs) in their testes.

According to specific embodiments, the subject is a prepubertal subject.

According to specific embodiments, the subject is infertile or at risk of becoming infertile.

According to specific embodiments, the subject has low sperm count or is azoospermic.

According to specific embodiments, the subject has non-obstructive azoospermia (NOA).

According to specific embodiments, the subject in need of aggressive chemotherapy and/or aggressive radiotherapy.

According to specific embodiments, the subject is a prepubertal male subject in need of aggressive chemotherapy and/or aggressive radiotherapy.

According to specific embodiments, the subject is diagnosed with cancer, such as a solid tumor or a non-solid cancer and/or cancer metastasis.

According to specific embodiments, the subject is a prepubertal male subject diagnosed with cancer, such as a solid tumor or a non-solid cancer and/or cancer metastasis.

For example, according to specific embodiments, the cancer is a hematological cancer.

According to specific embodiments, the cancer comprises a solid tumor.

Examples of cancer include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, testicular germ cells tumor, sacrococcygeal tumor, choriocarcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypemephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the subject is inflicted with cancer associated with male infertility.

Such cancers are known in the art and include, but not limited to acute myeloid leukemia (AML), brain tumor, extragonadal germ-cell tumor (EGGCTs), sarcoma, testicular germ cell tumor (TGCT), prostate cancer, skin cancer, Hodgkin lymphoma (HL), and non-Hodgkin lymphoma (NHL).

According to specific embodiments, the subject is diagnosed with autoimmune disease According to specific embodiments, the subject is a prepubertal male subject diagnosed with an autoimmune disease.

Autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March, 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. el al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March, 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infinte A J. And Kraig E, Int Rev Immunol 1999; 18 (1-2):83; Oshima M. et al., Eur J Immunol 1990 December; 20 (12):2563), neuropathies, motor neuropathies (Komberg A J. J Clin Neurosci. 2000 May; 7 (3):191); Guillain-Barre syndrome and autoimmune neuropathies (Kusunoki S. Am J Med Sci. 2000 April, 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; ¹²³ (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to specific embodiments, the subject is diagnosed with thalassemia (β-thalassemia major) According to specific embodiments, the subject is a prepubertal male subject diagnosed with thalassemia (β-thalassemia major).

According to specific embodiments, the subject is in need of bone marrow transplantation.

According to specific embodiments, the subject is a prepubertal male subject in need of bone marrow transplantation.

It should be noted that a testis tissue biopsy can be obtained from the subject using known surgical methods before administering the suitable anti-cancer drug(s) or the radiation therapy to the subject. In this case, the tissue biopsy can be cryopreserved and the tissue can be kept frozen until future use. Then after, upon thawing the frozen biopsy, the isolated spermatogonia are subjected to the in vitro proliferation and/or maturation method of some embodiments of the invention.

Additionally or alternatively, a testis tissue biopsy can be obtained from the subject during or after treating the subject with anti-cancer drug(s) (e.g., chemotherapy) or radiation. In this case, the tissue biopsy can be used as either a fresh biopsy for isolation of spermatogonia cells that are cultured in the methylcellulose culture system according to the method of some embodiments of the invention, or can be cryopreserved and kept frozen until future use. Then after, upon thawing the frozen biopsy, the isolated spermatogonia are subjected to the in vitro proliferation and/or maturation method of some embodiments of the invention.

As mentioned, qualifying the maturation stage, identifying and/or isolating mature cells following the in vitro culturing of the spermatogonium according to the method of some embodiments of the invention, can be done by detecting the expression of the markers typical to each cell type of the spermatogenesis, e.g., meiotic, postmeiotic and mature elongated sperm cell. Such markers are known in the art, and are also described herein above. Following is a non-limiting example of primary antibodies can be used to detect the spermatogenesis-specific markers:

(i) monoclonal mouse anti-human PLZF (promyelocytic leukemia zinc finger). Such antibodies are available for example from Santa Cruz;

(ii) Polyclonal goat anti-human GFR-α (GDNF family receptor alpha-1). Such antibodies are available for example from R&D Systems™, MN, USA;

(iii) Rabbit Polyclonal to Human SALL4 (spalt like transcription factor 4). Such antibodies are available for example from LSBio LifeSpan BioSciences, Inc. Seattle Wash., USA;

(iv) Polyclonal rabbit anti-human CD9 (CD9 molecules). Such antibodies are available for example from Abcam, Cambridge, UK;

(v) Polyclonal goat anti-human OCT4 (octamer-binding transcription factor 4). Such antibodies are available for example from Santa Cruz;

(vi) Polyclonal rabbit anti-human α-6-INTEGRIN. Such antibodies are available for example from Santa Cruz;

(vii) polyclonal rabbit anti-human VASA [also known as “DEAD-box helicase 4 (DDX4)” or “MVH”). Such antibodies are available for example from Santa Cruz, Calif., USA;

(viii) Polyclonal rabbit anti-human c-KIT (proto-oncogene receptor tyrosine kinase). Such antibodies are available for example from Dako, Calif., USA;

(ix) Polyclonal or monoclonal anti-BOULE. Such antibodies are available for example from Santa Cnz;

(x) Polyclonal or monoclonal anti-CREM-1. Such antibodies are available for example from Santa Cruz;

(xi) Polyclonal or monoclonal anti-ACROSIN. Such antibodies are available for example from Santa Cruz.

Exemplary secondary antibodies which can be used include, but are not limited to, Donkey anti-rabbit IgG (e.g. Cy3), Donkey anti-goat IgG (e.g. Cy3), and Goat anti-mouse IgG (e.g. Rhodamine red) Jackson Immuno Research (USA).

Non-limiting examples of suitable RT-PCR primers, which can be sued for detection of the spermatogenesis specific markers are provided in SEQ ID NOs: 11 and 12 (for OCT4); SEQ ID NOs: 13 and 14 (for SALL4); SEQ ID NOs: 15 and 16 (for alpha 6 integrin); SEQ ID NOs: 17 and 18 (for CD9); SEQ ID NOs: 19 and 20 (for GFR alpha-1); SEQ ID NOs: 21 and 22 (for c-KIT); SEQ ID NOs: 23 and 24 (for CREM); SEQ ID NOs: 25 and 26 (for protamine); SEQ ID NOs: 48 and 49 (for Acrosin); SEQ ID NOs: 50 and 51 (for Boule); SEQ ID NOs: 52 and 53 (for VASA); SEQ ID NOs: 54 and 55 (for PLZF).

Methods of Detecting the Expression Level of RNA

The expression level of the RNA in the cells of some embodiments of the invention can be determined using methods known in the arts.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

Oligonucleotide microarray—In this method oligonucleotide probes capable of specifically hybridizing with the polynucleotides of some embodiments of the invention are attached to a solid surface (e.g., a glass wafer). Each oligonucleotide probe is of approximately 20-25 nucleic acids in length. To detect the expression pattern of the polynucleotides of some embodiments of the invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting Expression and/or Activity of Proteins

Expression and/or activity level of proteins expressed in the cells of some embodiments of the invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxylin or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Addition of GM-CSF and/or Testosterone Increases Spermatogenesis in a 3D In Vitro Culture System

Granulocyte macrophages-colony stimulating factor (GM-CSF) is a hematopoietic growth factor that affects the development of hematopoietic cells from stem cells in the bone marrow. It was shown that GM-CSF is present in testicular macrophages and its receptor was demonstrated on the spermatozoa. It was shown to affect the viability of spermatogonial cells (Ghenima Dirami, Neelakanta Ravindranath., et al. “Effects of stem cell factor and granulocyte macrophage-colony stimulating factor on survival of porcine type A spermatogonia cultured in KSOM.” Biology of reproduction (1999). 61(1): 225-230).

Testosterone is a hormone that produces by Leydig cells in response to luteinizing hormone (LH) and is involved in the development of spermatogenesis under physiological conditions.

Experimental Methods

Tubular Cell Isolation—The procedure was performed under sterile conditions. Mice testes were removed and tunica albuginea was gently removed by scalpel knife. Seminiferous tubules (STs) were immersed with PBS and thereafter transported through a syringe 2-3 times to complete the mechanical digestion. Thereafter the STs were enzymatically digested as described by AbuMadigem et al. [International Journal of Molecular Sciences 19, 3804 (2018)].

Culturing of tubular cells—Seminiferous tubule cells were isolated from 7-days-old mice by enzymatic digestion as described hereinabove. Isolated cells were cultured in methylcellulose culture system (MCS) (42%) in the presence of 10% KnockOut serum replacement (KSR), and StemPro, EGF, FGF, LIF, GDNF (48%) (control medium). Fresh media were added again after two weeks of culture for additional two weeks. When indicated, the culture medium was further supplemented with GM-CSF (BioLegend, San Diego, Calif., USA, 0.1 ng/ml or 1 ng/ml), testosterone (Bayer, Hod hasharon, Israe, 10⁻⁷ M).

Expression of GM-CSF and GM-CSFR—Expression of GM-CSF and the GM-CSF receptor (GM-CSFR) was determined by qPCR analysis.

Expression of premeiotic and meiotic/postmeiotic markers—Expression of the premeiotic markers VASA, PLZF and the meiotic/postmeiotic markers BOULE and ACROSIN were determined by qPCR analysis using specific primers for each factor as the following:

VASA-
(SEQ ID NO: 52)
Fw-5′-AGTATTCATGGTGATCGGGAGCAG
and
(SEQ ID NO: 53)
Rw-5′-GCAACAAGAACTGGGCACTTTCCA;
PLZF-
(SEQ ID NO: 54)
Fw-5′-AGCTTGAAATACGTGGCCAGA
and
(SEQ ID NO: 55)
Rw-5′-TGAGCAGTTCACACTTCATCCC;
Boule-
(SEQ ID NO: 50)
Fw-5′-AACCCAACAAGTGGCCCAAGATAC
and
(SEQ ID NO: 51)
Rw-5′-CTTTGGACACTCCAGCTCTGTCAT;
ACROSIN-
(SEQ ID NO: 48)
Fw-5′-TGTCCGTGGTTGCCAAGGATAACA
and
(SEQ ID NO: 49)
Rw-5′-AATCCGGGTACCTGCTTGTGAGTT.

Experimental Results

The present inventors uncovered that GM-CSF is produced by Sertoli cells, Leydig cells, premeiotic cells (CDH1 positive cells) and meiotic cells (BOULE positive cells) (data not shown); and that the GM-CSF receptor (GM-CSFR) is also present on Testicular somatic cells and developed germ cells (data not shown).

To test the effects of GM-CSF and testosterone on in vitro development of spermatogenesis, isolated seminiferous tubule cells were cultured in MCS in the presence of a medium supplemented with GM-CSF and/or testosterone and the levels of several premeiotic and meiotic/postmeiotic markers were determined following 4 weeks of culture, as compared to seminiferous tubule cells cultured under the same conditions but without GM-CSF and/or testosterone (control).

Addition of GM-CSF (0.1 or 1 ng/ml) to isolated seminiferous tubule cells significantly increased the expression level of the premeiotic markers VASA (FIG. 1B) and PLZF (FIG. 1C) and the percentage of VASA positive cells premeiotic after 4 weeks of culture compared to control cultured cells. The addition of testosterone after 2 weeks of culturing in control medium for additional 2 weeks significantly increased the expression levels of VASA and PLZF (FIGS. 1B and 1C, respectively) but did not affect the percentages of VASA positive cells (FIG. 1A). The addition of testosterone for two weeks after two weeks culturing in the presence of GM-CSF did not affect the percentages of VASA cells compared to GM-CSF only (FIG. 1A), but significantly decreased the expression levels of VASA and PLZF compared to GM-CSF only (FIGS. 1B and 1C; respectively). It should be noted that isolated seminiferous tubule cells prior to culturing (pre-cult) contain VASA and PLZF cells since at this age (7-days old) the mice contain premeiotic cells.

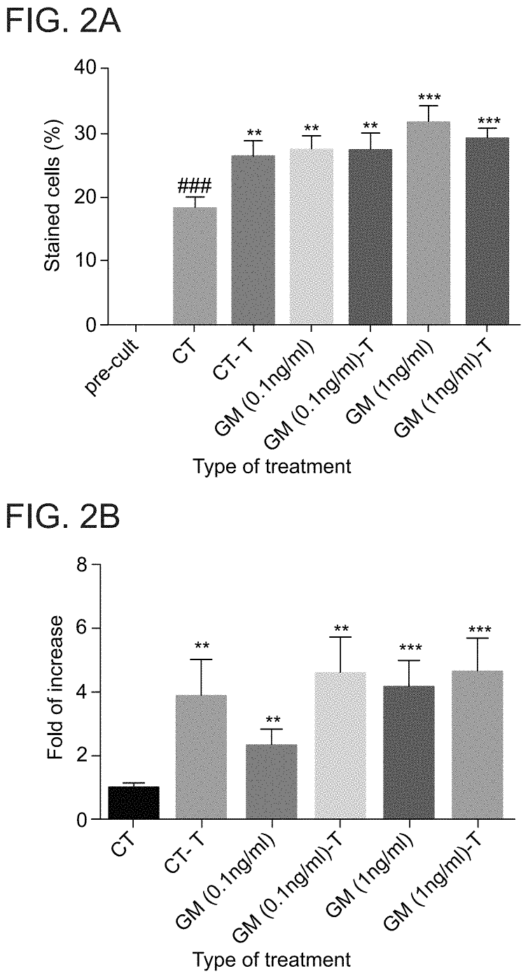

Further, addition of GM-CSF (0.1 or 1 ng/ml) to isolated seminiferous tubule cells significantly increased the expression levels of the meiotic marker BOULE (FIG. 2B) and the percentages of BOULE positive cells (FIG. 2A) after 4 weeks of culture compared to control cultured cells. The addition of testosterone after 2 weeks of culturing in control medium for additional 2 weeks significantly increased BOULE expression levels (FIG. 2B) and the percentages of BOULE positive cells (FIG. 2A). Also, addition of testosterone for two weeks after two weeks of culturing in the presence of GM-CSF did not affect the percentages of BOULE positive cells nor BOULE expression levels compared to GM-CSF only (FIGS. 2A-B). It should be notes that isolated seminiferous tubule cells prior to culturing (pre-cult) did not contain boule cells since at this age (7-days old) the mice do not develop meiotic cells.

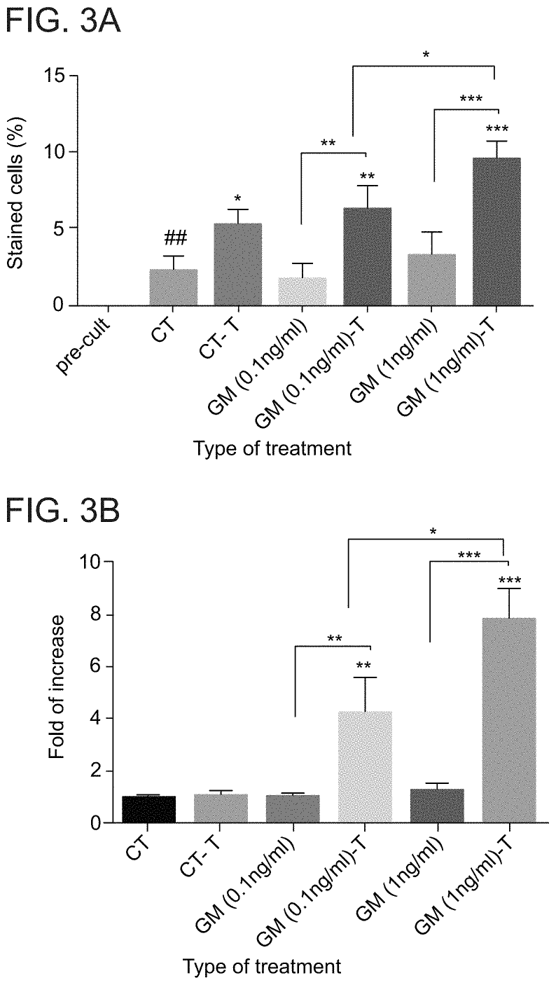

Moreover, as is further shown in FIGS. 3A-B, addition of GM-CSF (0.1 or 1 ng/ml) to isolated seminiferous tubule cells did not significantly increase the expression levels of the meiotic/postmeiotic marker acrosin (FIG. 3B) nor the percentage of acrosin positive cells (FIG. 3A) after 4 weeks of culture compared to control cultured cells. Addition of testosterone after 2 weeks of the culturing in control medium for additional 2 weeks significantly increased the percentages of acrosin positive cells (FIG. 3A), but did not affect acrosin expression levels compared to cultrol cultured cells (FIG. 3B). Addition of testosterone for two weeks after two weeks culturing in the presence of GM-CSF significantly increased both the percentages of acrosin positive cells and acrosin expression levels postmeiotic compared to GM-CSF treatment only (FIGS. 3A-B). It should be noted that isolated seminiferous tubule cells prior to culturing (pre-cult) did not contain acrosin cells since at this age (7-days old) the mice do not develop meiotic/postmeiotic cells.

Overall, these results show for the first time a direct effect of GM-CSF on the development of spermatogenesis in vitro using methylcellulose and 3D in vitro culture system (FIGS. 1A-3B). And more specifically, these results demonstrate for the first time that addition of GM-CSF to the 3D in vitro culture increases the development of premeiotic and meiotic cells, but not acrosin (meiotic/postmeiotic cell marker) positive cells. Addition of testosterone after two weeks of culture increases the development of premeiotic, meiotic and postmeiotic cells. On the other hand, addition of testosterone two weeks after the addition of GM-CSF significantly increases the development of acrosin positive cells compared to GM-CSF or testosterone alone.

### Example 2

### Involvement of Cytokines and Hormones in the Development of Spermatogenesis In Vitro from Spermatogonial Cells of Cyclophosphamide-Treated Immature Mice

The harmful effect of chemotherapy/radiotherapy on prepubertal and pubertal male fertility is well recognized^{51,52}. While fertility preservation of male pubertal patients is possible by sperm cryopreservation before chemotherapy/radiotherapy, the fertility preservation strategies for prepubertal cancer patients, who do not yet produce sperm, is still experimental^{38-43,53}. In addition, most of the studies to evaluate the effect of chemotherapy/radiotherapy on the development of spermatogenesis and testicular cell functionality were performed in adult rodents⁵⁴⁻⁵⁸. On the other hand, the cellular components and functionality of the testis of immature and adult males are different, and thus the effect of chemotherapy/radiotherapy on these cells could be different.

In the present study the present inventor examined the in vivo effect of cyclophosphamide, an anti-cancer chemotherapy used in prepubertal patients, on the development of spermatogenesis in immature mice, and the possible use of the survivor spermatogonial cells to develop spermatogenesis in vitro using 3D MCS.

Experimental Procedures

Animals—All experiments were performed in accordance with the Guiding Principles for the Care and Use of Research Animals Promulgated by the Society for the Study of Reproduction. It was confirmed by the Ben-Gurion University Ethics Committee for Animal Use in Research (IL-17-11-2014). Sexually immature 7-days-old ICR male mice were purchased from Envigo Laboratories, Jerusalem, Israel.

Cyclophosphamide administration—Cyclophosphamide (CP) powder (MP Biomedicals, Illkirch, France) was dissolved in sterile PBS. 100 μl of CP (100 mg/kg) (CP group) or 100 μl PBS (control group; CT) were intraperitoneally (i.p) injected into the immature mice. Injections were performed once a week for 3 weeks. One week to 5 weeks post the last injection (post-treatment) mice were sacrificed by CO₂. Testes were weighed and fixed in methanol or Bouins' solution for histological evaluation and/or frozen in −70° C. for RNA extraction or were immediately used for tubular cell isolation, cellular evaluations and in vitro culture in MCS.

Tubular Cell Isolation—The procedure was performed under sterile conditions. Testes from both control and CP-treated mice were removed and tunica albuginea was gently removed by scalpel knife. Seminiferous tubules (STs) were immersed with PBS and thereafter transported through a syringe 2-3 times to complete the mechanical digestion. Thereafter the STs were enzymatically digested as described by AbuMadigem et al. [International Journal of Molecular Sciences 19, 3804 (2018)].

In vitro Culture of Tubular Cells—Cells were seeded in 24-wells plates and cultured in methylcellulose culture system (MCS). Each well contained 200,000 cells/500 μl suspended in media-containing StemPro (33%) (Gibco, USA), KnockOut serum replacement-KSR (25%) (Gibco, USA), growth factors, penicillin-streptomycin (pen-strep, 1%) and methylcellulose (MC; 42%) (R&D Systems™, Minneapolis, USA) as described by AbuMadigem et al. [International Journal of Molecular Sciences 19, 3804 (2018)]. When indicated the media was further supplemented with follicle stimulating hormone (FSH) (7.5 IU/ml) (Serono, Switzerland), interleukin 1 alpha (IL-1α) (20 pg/ml) (BioLegend®) or tumor necrosis factor alpha (TNF-α) (20 pg/ml) (BioLegend®) from the beginning of the culture. Further, when indicated, testosterone (10⁻⁷ M) (Bayer, Israel) alone or in combination with other factors (FSH, IL-1α or TNFα) was added at the last week of the culture. Fresh media and growth factors were added every 7-10 days in a ×10 concentration (50 μl/well). The cells were usually cultured in MCS for 4-5 weeks.

Histological and immunostaining of testicular tissues and cells—Slide preparation and fixation of testicular tissues and cells, immunostaining and hematoxylin-eosin tissue staining were performed as described by AbuMadigem et al. [International Journal of Molecular Sciences 19, 3804 (2018)]. Following removal of the blocking buffer, the following first antibodies were added: monoclonal mouse anti mouse Vimentin (Novus, 1:500), polyclonal goat anti mouse α-sma (Abcam, 1:250), polyclonal goat anti-mouse α-6 Integrin (Santa Cruz, Calif., USA; 1:40), polyclonal rabbit anti-mouse VASA (Santa Cruz, Calif., USA; 1:100), polyclonal rabbit anti-mouse CD9 (Santa Cruz, Calif., USA; 1:100), monoclonal mouse anti-mouse c-KIT (Santa Cruz, Calif., USA; 1:50), monoclonal mouse anti-mouse GFR-α1 (Santa Cruz, Calif., USA; 1:50), polyclonal rabbit anti-mouse CREM-1 (Santa Cruz, Calif., USA; 1:50), polyclonal rabbit anti-mouse BOULE (Santa Cruz, Calif., USA; 1:50), polyclonal rabbit anti-mouse ACROSIN (Santa Cruz, Calif., USA; 1:200). Following overnight incubation at 4° C., the slides were washed and the specific secondary antibodies were added compatibly to the first antibodies [goat anti-mouse IgG (Rhodamine red), donkey anti-goat IgG (Cy3); Jackson Immuno Research (West Grove, Pa., USA) for 40 minutes at room temperature. After washing, the slides were dried and DAPI, which stains the nuclei blue, was added to the tissues, and cover slides were applied. Staining in blocking buffer without the first antibody served as a negative control. Results were quantified by counting the positive-stained cells for each marker compared to the total no. of cells in the counted field. Data is presented as percentage of positive-stained cells.

Microscope analysis—Preformed by Olympus IX70 microscope (Olympus, Novato, Calif., USA). Digital images were prepared using Image-Pro Plus (Media Cybernetics, Bethesda, Md., USA), Microsoft Excel, and Adobe Photoshop 7.0 software.

Gene expression—RNA was extracted from isolated testicular cells, from control or CP-treated immature mice, by GenElute Mannalian Total RNA Miniprep Kit [Sigma, St. Louis, USA). cDNA synthesis was performed according to the qScript cDNA Synthesis Kit (Quantabio, Beverly, Mass., USA) and qPCR was performed using specific primers for each examined marker: ABP (forward: GCAGCATGAGGATTGCACTA (SEQ ID NO: 1); reverse: CATGAGGCTGGGGAATGTCT (SEQ ID NO:2); product size, 237 bp), INHIBIN B (forward: CCTGTCATCAGGGCAAGTGA (SEQ ID NO:3); reverse: TCGAGGCAGACGCCTTATTC (SEQ ID NO:4); product size, 209 bp), FSH-R (forward: GTGCATTCAACGGAACCCAG (SEQ ID NO:5); reverse: AGGGAGCTTITTCAAGCGGT (SEQ ID NO: 6); product size, 206 bp), Transferrin (forward: CCAAGCTCCAAACCATGTTGT (SEQ ID NO:7); reverse: ACAGATTGCATGTACTCCGCT (SEQ ID NO:8); product size: 231 bp), Housekeeping gene GAPDH (forward: ACCACAGTCCATGCCATCAC (SEQ ID NO:9); reverse: CACCACCCTGTTGCTGTAGCC (SEQ ID NO:10); product size, 450 bp). qPCR reaction was performed following the 2× qPCRBIO SyGreen Blue Mix Hi-ROX (PCR Biosystems Ltd, Aztec House, 397-405 Archway Road, London, UK) protocol and was performed using the LightCycler 96 real-time PCR machine (Roche, Roche Diagnostics Corporation, Roche CustomBiotech, Indianapolis, Ind., USA). Program: preincubation 10 minutes at 95° C., 40 cycles of 15 seconds at 95° C., 15 seconds at 60° C. and 10 seconds at 72° C. Melting cycle: 10 seconds at 95° C., 60 seconds at 65° C., and 1 second at 97° C. PCR products were identified by the melting curve. The “threshold cycle” (Ct) value for each transcript was identified. The relative quantity of gene expression was analyzed by the 2° method. Results were expressed as the fold of increase related to the GAPDH of the same examined sample and relatively compared to control treatment group.

Experimental Results

The Effects of Treatment of Immature Mice with Cyclophosphamide (CP) on Mice Testes and Spermatogenic Cells:

Treatment of immature mice with CP significantly decreased (p<0.001) their testicular weight at all time points tested during 5 weeks after the last injection compared to control mice (FIG. 4A). CP treatment also impaired seminiferous tubules histology during 3 weeks after the last injection (FIG. 4B). Specifically, the germinal epithelium was decreased and the seminiferous tubules appeared empty of most of the cells 1 and 3 weeks post treatment (FIG. 4B), wherein the most severe damage for the seminiferous tubules was observed 1-2 weeks post-treatment (FIG. 4C). Starting 3 weeks post treatment onwards, testis histology was gradually restored, and five weeks post-treatment the histology of the seminiferous tubules was similar to the control group (FIGS. 4B-C). When tested at day 10 post-treatment, CP treatment of immature mice significantly impaired seminiferous tubules histology (FIG. 4D), decreased the testicular weight (FIG. 4E; p<0.001) and testicular cell count (FIG. 4F; p<0.001) compared to control.

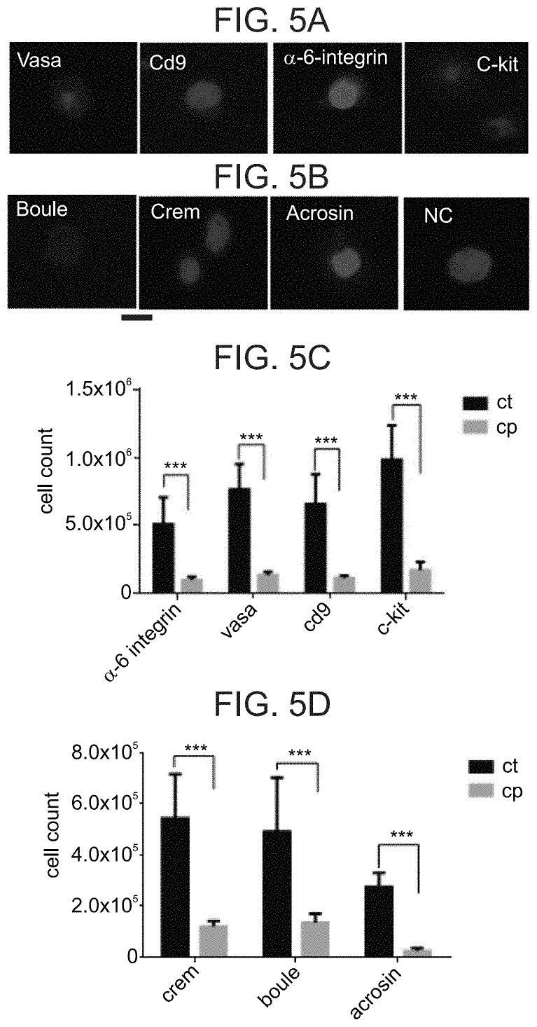

Following, the effect of CP on the number of subpopulation of spermatogenic cells (premeiotic and meiotic/postmeiotic cells) isolated from seminiferous tubules of immature mice was examined 10-days post the last CP-injection using immunofluorescence staining for markers specific for each type of cells [premeiotic cells (FIGS. 5A and 5C) and meiotic/postmeiotic cells (FIGS. 5B and 5D)]. The results show that CP treatment significantly reduced the number of premeiotic cells stained positive to α-6 INTEGRIN, VASA, CD9 and c-KIT compared to control (FIG. 5C; p<0.001); and the number of meiotic cells stained positive to CREM and BOULE and to the meiotic/postmeiotic marker, acrosin, compared to control (FIG. 5D; p<0.001).

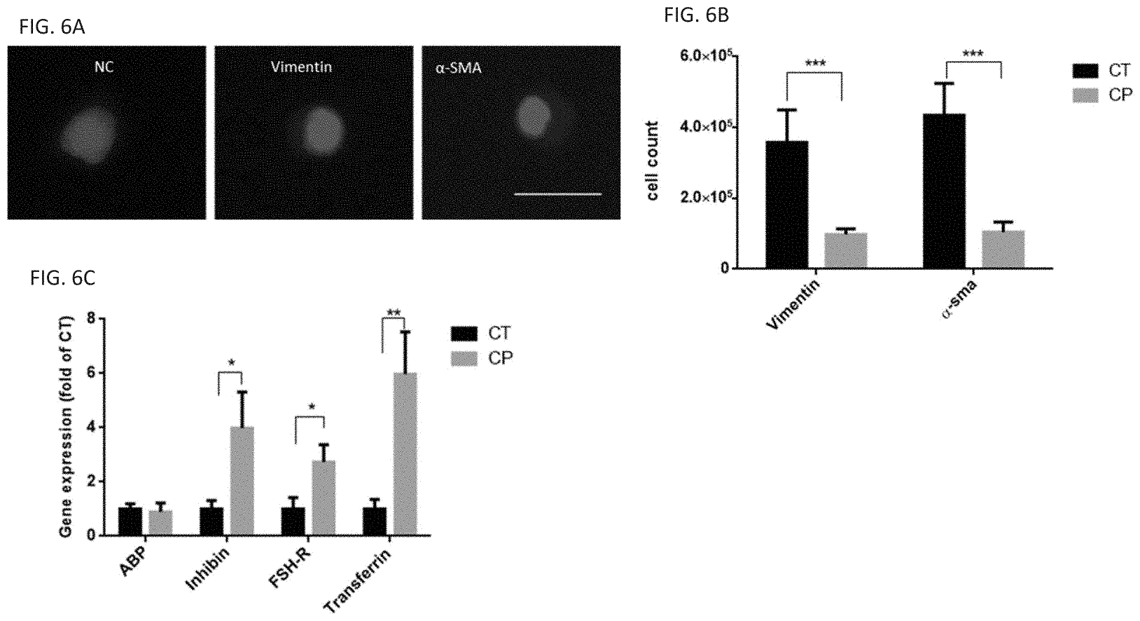

Moreover, the effect of CP on the number of Sertoli and peritubular cells isolated from seminiferous tubules of immature mice was examined 10-days post last CP-injection, using immunofluorescence staining for markers specific for each type of cells (vimentin—a marker of Sertoli cells and αSMA—a marker of peritubular cells) (FIGS. 6A-B). The results show that CP treatment significantly reduced the number of Sertoli and peritubular cells compared to control (FIG. 6B; p<0.001). Evaluating the effect of CP on the functionality of Sertoli cells by examining the expression levels of some functional factors (known to affect spermatogenesis) produced by Sertoli cells, show that CP treatment significantly increased the expression levels of inhibin, FSH-receptor (FSH-R) and transferrin compared to control (FIG. 6C; p<0.05, 0.05 and 0.01, respectively), but did not significantly affect the expression levels of androgen binding protein (ABP) compared to control (FIG. 6C). These changes in expression levels may change the cell-cell interactions in the seminiferous tubules and thus to impair the process of spermatogenesis and may lead to subfertility or infertility.

Taken together, treatment of immature mice with CP significantly decreases testicular weight, impairs seminiferous tubule normal histology and decreases the number of subpopulation of spermatogenic cells and Sertoli cells. In addition CP affects the functionality of Sertoli cells, and thus may affect the microenvironment surrounding the testicular germ cells. These results indicate that testicular germ cells of immature mice are very sensitive to the effect CP even though some of these cells survive the CP treatment. This may lead to impairment in the survival, proliferation and differentiation of spermatogonial cells and thus to subfertility or infertility. Without being bound by theory, the reduction in meiotic and postmeiotic cells following CP treatment could be related to the reduction in the numbers of testicular dividing germ cells (premeiotic cells that mitotically divide), and also to direct effect of CP on the meiotic cells.

Effect of Hormones (FSH and Testosterone) and Cytokines (IL-1α and TNFα) on the Proliferation and Differentiation of Spermatogonial Cells from CP-Treated Immature Mice In Vitro in MCS:

Ten days after the last injection of CP, testes were removed, seminiferous tubules were separated, and cells were enzymatically isolated and cultured in MCS. In some wells, IL1-α, TNF-α or FSH were added from the beginning of the culture. Further, in some wells testosterone was added at the last week of the culture, alone or in combination with IL-1α, TNF-α or FSH. Every 10-14 days, new media containing the same composition of factors was added to the culture. After 4-5 weeks, the developed colonies and cells were collected, and the cells were fixed by cold methanol and stained by immunofluorescence staining using specific antibodies for markers of the pre meiotic (VASA, CD9, GFRα, α-6 INTEGRIN, c-KIT), meiotic (CREM, BOULE) and meiotic/postmeiotic (acrosin) cells and the percentage of cells stained for each examined marker in each treatment was evaluated and compared between the different groups. The results are summarized in Table 1 hereinbelow.

As described hereinabove, isolated cells from seminiferous tubules of immature mice ten post CP treatment still have spermatogenic cells that were positively stained for premeiotic (VASA, CD9, GFR-a, a-6-INTEFRIN, and c-KIT), meiotic (BOULE AND CREM) and to meiotic/postmeiotic (ACROSIN) markers prior to beginning of the culture (FIGS. 5A-B and Table 1 hereinbelow). Proliferation (production of colonies; FIGS. 7A-F) and differentiation (a significant increase in the number of c-KIT and ACROSIN positive cells) of the spermatogonial cells was induced in vitro in MCS, compared to prior to the beginning of culture (Table 1 hereinbelow). Specifically, in-vitro culture of the isolated cells in MCS maintained the percentage of the premeiotic VASA, CD9 and GFR-α positive cells, but significantly decreased the percentage of a-6-INTEGRIN positive cells compared to before culture (Table 1 hereinbelow). On the other hand, the percentage of the meiotic cells expressing BOULE and CREM did not change compared to before culture (Table 1 hereinbelow). Further, the percentage of the meiotic/postmeiotic ACROAIN-positive cells was significantly increased in MCS compared to before culture (Table 1 hereinbelow).

Addition of FSH to the culture medium did not show any significant effect on the percentage of premeiotic, meiotic and meiotic/pos-tmeiotic examined cells developed in MCS compared to cells cultured in control medium (Table 1 hereinbelow).

Addition of testosterone to the culture medium significantly increased the percentage of the premeiotic CD9 positive cells and the meiotic/postmeiotic ACROSIN positive cells, without any significant effect on the other examined premeiotic and meiotic cells developed in MCS compared to cells cultured in control medium (Table 1 hereinbelow). Addition of both FSH and testosterone to the culture medium significantly decreased the percentage of the premeiotic CD9 positive cells compared to addition of testosterone alone, and the meiotic/postmeiotic ACROSIN positive cells developed in MCS compared to addition of FSH or testosterone only (Table 1 hereinbelow). The addition of FSH and testosterone did not significantly affect the percentage of the other examined premeiotic and meiotic cells developed in MCS compared to addition of FSH or testosterone only (Table 1).

Addition of IL-1α to the culture medium did not show any significant effect on the percentage of the examined premeiotic, meiotic and meiotic/postmeiotic cells developed in MCS compared to cells cultured in control medium, under these conditions (Table 1 hereinbelow). However, addition of both IL-1α and testosterone to the culture medium significantly increased the percentage of the premeiotic VASA positive cells and the meiotic BOULE positive cells developed in MCS compared to addition of IL-1α or testosterone only, without any significant effect on the other examined premeiotic and meiotic/postmeiotic cells developed in MCS compared to testosterone alone (Table 1 hereinbelow).

Addition of TNFα to the culture medium significantly increased the percentage of the premeiotic CD9 positive cells, without any significant effect on the other examined premeiotic and meiotic or meiotic/postmeiotic positive cells developed in MCS compared to cells cultured in control medium, under these conditions (Table 1 hereinbelow). Interestingly, addition of both TNFα and testosterone to the culture medium significantly decreased the percentage of the meiotic/postmeiotic ACROSIN positive cells without any significant effect on the other examined premeiotic or meiotic cells developed in MCS compared to additional of testosterone alone (Table 1 hereinbelow).

TABLE 1
Effect of hormones or cytokines on the in vitro development of spermatogonial
cells isolated from CP-treated immature mice

| | Spermatogenic markers | | | | | | | |
|---|---|---|---|---|---|---|---|---|
| | Pre-meiotic | | | | | | | |
| | | | | a-6- | | Meiotic/postmeiotic | | |
| Treatment | VASA | CD9 | GFR-a | INTG | C-KIT | BOULE | CREM | ACROSIN |
| BC | 23.65 ± | 14.88 ± | 13.1 ± | 28.05 ± | 6.65 ± | 31.25 ± | 20.8 ± | 10.3 ± |
| | 5.79 | 4.86 | 2.33 | 7.93 | 1.25 | 9.5 | 6.2 | 1.65 |
| AC | 26.23 ± | 19.56 ± | 21.77 ± | 14.86 ± | 19.5 ± | 19.16 ± | 25.46 ± | 35.96 ± |
| | 4.26 | 1.44 | 1.9 | 5.88 * | 1.68 * | 4.29 | 7. 51 | 5.9 *** |
| AC + FSH | 24.04 ± | 25.62 ± | 18.97 ± | 18.8 ± | 22.42 ± | 20.6 ± | 35.16 ± | 43.92 ± |
| | 4.42 | 2.38 | 5.7 | 5.47 | 5.39 | 4.09 | 6.46 | 5.65 |
| AC + T | 23.76 ± | 42.63 ± | 24.03 ± | 12.23 ± | 11.13 ± | 22.15 ± | 38.42 ± | 55.53 ± |
| | 3.56 | 5.19 | 4.22 | 0.76 | 2.09 | 2.29 | 6.47 | 6.86 ## |
| | | ### | | | | | | |
| AC + | 21.15 ± | 21.02 ± | 20.77 ± | 20.87 ± | 16.9 ± | 27.03 ± | 34.45 + | 25.67 ± |
| FSH + T | 2.02 | 4.56 | 4.36 | 3.17 | 2.91 | 8.39 | 6.2 | 6.01 $$$, |
| | | $$$ | | | | | | @@ |
| AC + IL-1 | 20.8 ± | 23.4 ± | 22.77 ± | 17.5 ± | 16.5 ± | 27.16 ± | 30.45 + | 34.27 ± |
| | 5.33 | 6.14 | 4.23 | 7.44 | 4.05 | 6.64 | 8.12 | 10.77 |
| AC + | 49.2 ± | 48.36 ± | 25.2 ± | 17.93 ± | 20.1 ± | 61.12 ± | 42.82 ± | 55.09 ± |
| IL-1 + T | 8.3 $, | 6.43 | 6.57 | 3.16 | 5.83 | 5.1 $$$, | 5.12 | 7.61 |
| | @ | | | | | @@@ | | |
| AC + TNF | 31.67 ± | 33.52 ± | 25.3 ± | 25.76 ± | 29.05 ± | 21.1 ± | 36.1 ± | 36.4 ± |
| | 6.16 | 4.29 # | 4.1 | 4.61 | 5.51 | 4.01 | 9.1 | 7.32 |
| AC + | 28.92 ± | 43.63 ± | 27.2 ± | 34.9 | 27.7 ± | 16.76 ± | 35.1 ± | 34.52 ± |
| TNF + T | 11.24 | 11.03 | 10.9 | | 4.4 | 6.16 | 16.58 | 10.79 $$ |

BC—before culture; AC—after culture in control media; AC+—after culture in media supplemented with the indicated hormone/cytokine; T—testosterone, IL-1—IL1α; TNF—TNFα.
*—Compared to BC (*—p < 0.05; **—p < 0.01; ***—p < 0.001); #—Compared to AC (#—p < 0.05; ##—p < 0.01; ###—p < 0.001); $—Compared to T ($—p < 0.05; S$—p < 0.01; $$$—p < 0.001); @—Compared to FSH or IL-1 or TNF (according to the pair) (@—p < 0.05; @@—p < 0.01; @@@—p < 0.001).
Bold—Significant increase.
Underlined—Significant decrease.

### Example 3

### Addition of IL-1 Alpha Increases Spermatogenesis in a 3D In-Vitro Culture System

Interleukin-1 (IL-1) is a family of cytokines produced by most of the body cells. This family is composed of different factors including IL-1 alpha (IL-1α), IL-1 beta (IL-1b) and IL-1 receptor antagonist (IL-1ra). These factors affect target cells by binding to IL-1 receptor type I (IL-1RI). There is an additional receptor, IL-1RII, which is working as a decoy and thus regulates the effect of IL-1 on the target cells. In addition, IL-1ra binds to IL-1RI but do not induce signals into the cells, thus it also regulates the effect of IL-1a and IL-1b on their receptor. The present inventor's research group and others showed that IL-1 family is present in the different cell types of the testis, including Leydig, Sertoli, peritubular and germ cells (different types through their differentiation). It was suggested that IL-1 is involved in the proliferation of testicular germ cells (Huleihel, M., Abu Elhija, M., and Lunenfeld, E. 2007. In nitro culture of testicular germ cells: regulatory factors and limitations. Growth Factors. 25: 236-252; Huleihel, M., and Lunenfeld, E. 2004. Regulation of spermatogenesis by paracrine/autocrine testicular factors. Asian J. Androl. 6: 259-268; Huleihel, M., and Lunenfeld, E. 2002. Involvement of intratesticular IL-1 system in the regulation of Sertoli cell functions. Mol. Cell. Endocrinol. 187: 125-132).

Experimental Procedures

Culturing of tubular cells—Seminiferous tubule cells were isolated from 7-days-old mice by enzymatic digestion as described in Examples 1 and 2 hereinabove. Isolated cells were cultured in methylcellulose culture system (MCS) (42%), in the presence of 10% KSR, and StemPro, EGF, FGF, LIF, GDNF (48%) (control medium). Fresh media were added again after two weeks of culture for additional two weeks. When indicated, the culture medium was further supplemented with IL-1α (10 pg/ml, PeproTech, Rehovot, Israel, Cat #211-11A-50UG) from the beginning of the culture and also after two weeks of culture for additional two weeks or only after two weeks of the culture for additional two weeks.

Immunostaining—Cells and clusters/colonies developed in the culture were collected after 4 weeks and examined for the presence of the premeiotic cells (VASA), or the meiotic cells (BOULE) or the Meiotic/postmeiotic cells (ACROSIN) by immunofluorescence staining (IF) using antibodies specific for each marker. Also, cells before culture (BC) were examined for the premeiotic, meiotic and postmeiotic stages. The following primary antibodies were used: Rabbit Polyclonal anti-VASA (NOVUS biologicals, Littleton, Colo. NBP2-24558), mouse monoclonal anti-boule (Santa Cruz Biotechnology, sc-166660) and Rabbit polyclonal anti-Acrosin (NOVUS biologicals, NBP-14260). Results were quantified by counting the positive-stained cells for each marker compared to the total no. of cells in the counted field. Data is presented as percentage of positive-stained cells.

Experimental Results

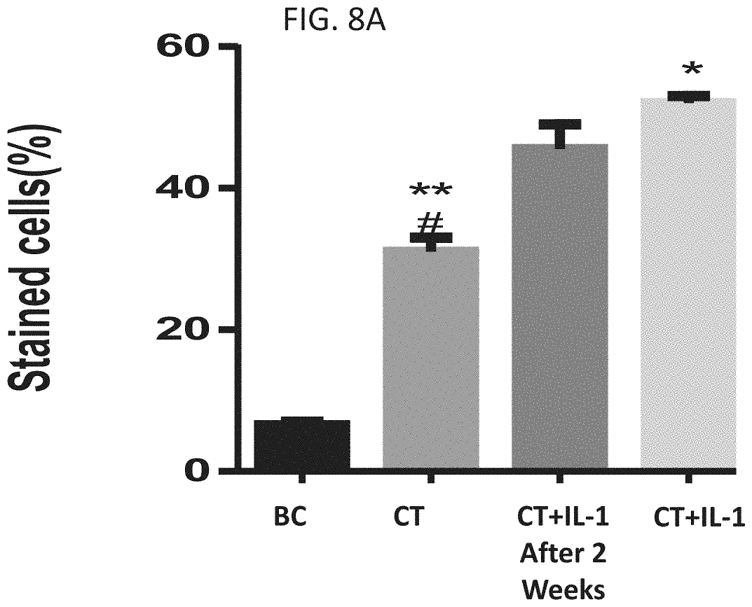

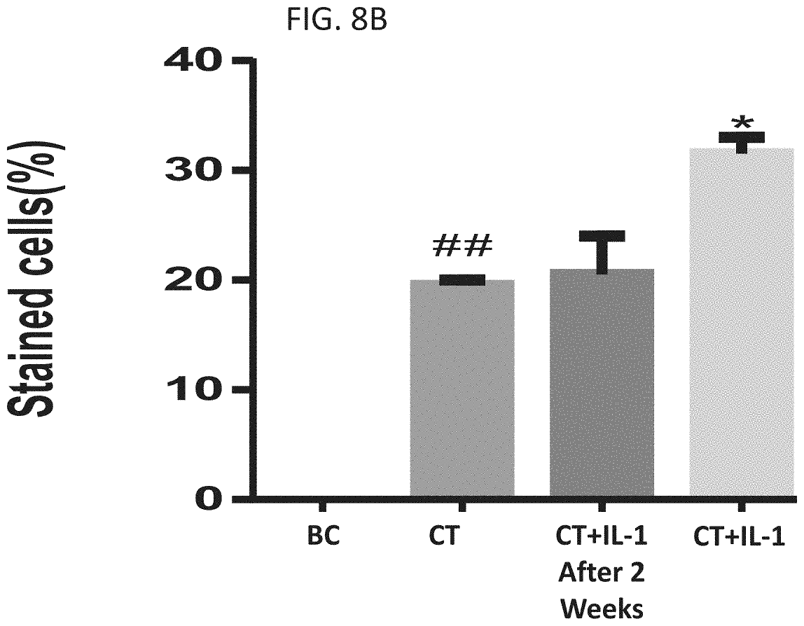

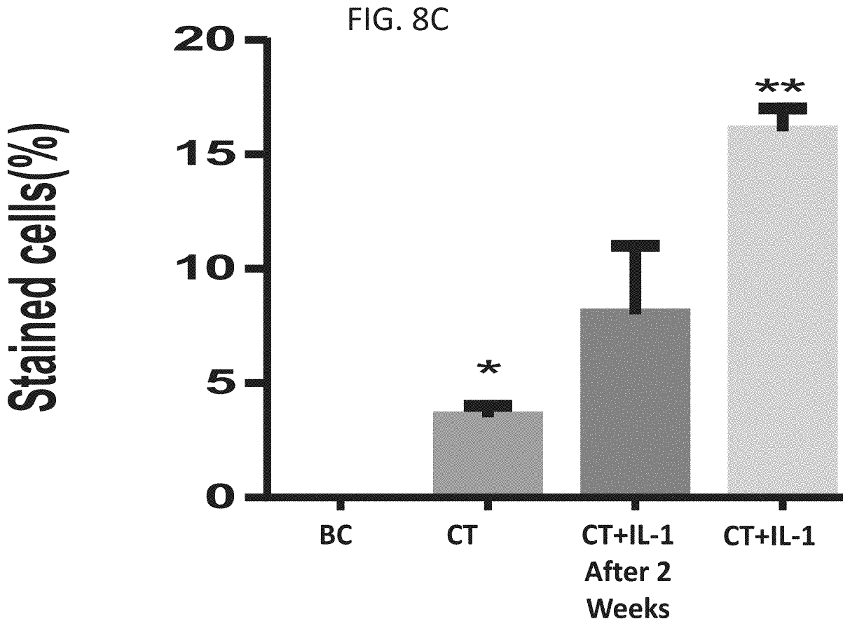

The results show a direct effect of IL-1α (interleukin 1 alpha) on the development of spermatogenesis in vitro using methylcellulose and 3D in vitro culture (FIGS. 8A-C).

Specifically, addition of IL-1α (10 pg/ml) to isolated seminiferous tubule cells significantly increased the percentages of VASA positive cells after 4 weeks of culture compared to control medium (FIG. 8A). Further, addition of IL-1α after 2 weeks of the culture for additional 2 weeks also increased the percentages of VASA positive cells, but not significantly compared to control medium (FIG. 8A). Of note, the control conditions that contain different growth factors significantly increased the percentages of VASA positive cells compared to before culture (FIG. 8A).

Furthermore, addition of IL-1α (10 pg/ml) to isolated seminiferous tubule cells significantly increased the percentages of BOULE positive cells (meiotic cells) after 4 weeks of culture compared to control medium (FIG. 8B). Addition of IL-1 after 2 weeks of the culture for additional 2 weeks also increased the percentages of BOULE positive cells, but not significantly compared to control medium (FIG. 8B). Of note, the control conditions that contain different growth factors increased the percentages of BOULE positive cells compared to before culture (FIG. 8B), wherein before culture cells did not contain BOULE positive cells since at this age (7-days-old) the mice do not develop meiotic cells.

Moreover, addition of IL-1α (10 pg/ml) to isolated seminiferous tubule cells significantly increased the percentages of acrosin positive cells (meiotic/postmeiotic cells) after 4 weeks of culture compared to control medium (FIG. 8C). Addition of IL-1α after 2 weeks of the culture for additional 2 weeks also increased the percentages of acrosin cells, but not significantly compared to control medium (FIG. 8C). Of note, the control conditions that contains different growth factors increased the percentages of acrosin positive cells compared to before culture (FIG. 8C), wherein before culture cells did not contain acrosin cells since at this age (7-days-old) the mice do not develop meiotic/postmeiotic cells.

### Example 4

### Addition of IL-1 Beta Increases Spermatogenesis in a 3D In-Vitro Culture System

Experimental Procedures

Culturing of tubular cells—Seminiferous tubule cells were isolated from 7-days-old mice by enzymatic digestion as described in Examples 1 and 2 hereinabove. Isolated cells were cultured in methylcellulose culture system (MCS) (42%), in the presence of 10% KSR, and StemPro, EGF, FGF, LIF, GDNF (48%) (control medium). When indicated, the culture medium was further supplemented with IL-1β (1, 10 or 100 pg/ml, PeproTech, Rehovot, Israel, Cat #221-11B-50UG). Fresh media were added again after two weeks of culture for additional two weeks.

Immunostaining—As described in Example 3 hereinabove.

Experimental Results

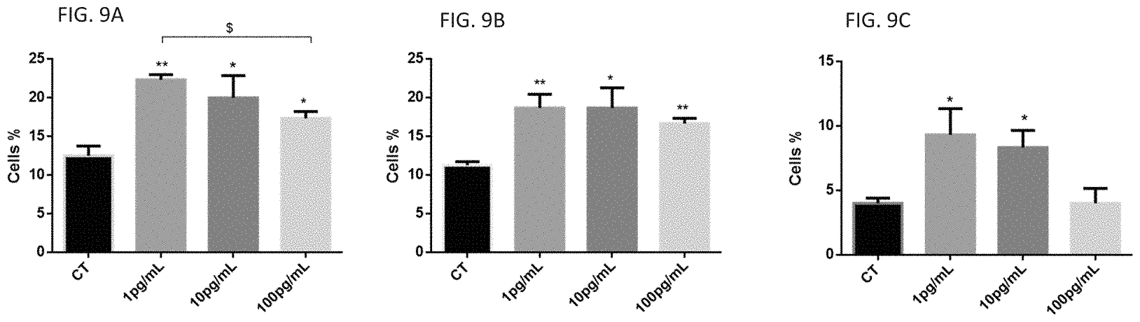

The results show a direct effect of IL-1β (interleukin 1 beta) on the development of spermatogenesis in vitro using methylcellulose and 3D in vitro culture (FIGS. 9A-C). Specifically, addition of IL-1β to isolated seminiferous tubule cells significantly increased the percentages of VASA positive cells (FIG. 9A), BOULE positive cells (FIG. 9B) and acrosin positive cells (FIG. 9C) after 4 weeks of culture compared to control medium.

### Example 5

### Addition of IL-6 Increases Spermatogenesis in a 3D In-Vitro Culture System

Experimental Procedures

Culturing of tubular cells—Seminiferous tubule cells were isolated from 7-days-old mice by enzymatic digestion as described in Examples 1 and 2 hereinabove. Isolated cells were cultured in methylcellulose culture system (MCS) (42%), in the presence of 10% KSR, and StemPro, EGF, FGF, LIF, GDNF (48%) (control medium). When indicated, the culture medium was further supplemented with IL-6 (1, 10 or 100 pg/ml, PeproTech, Rehovot, Israel, Cat #216-16-50UG). Fresh media were added again after two weeks of culture for additional two weeks.

Immunostaining—As described in Example 3 hereinabove.

Experimental Results

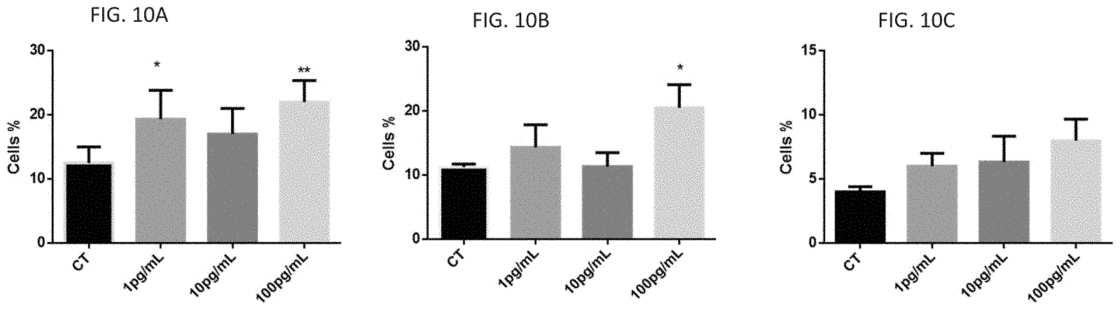

The results show a direct effect of IL-6 on the development of spermatogenesis in vitro using methylcellulose and 3D in vitro culture (FIGS. 10A-C). Specifically, addition of IL-6 to isolated seminiferous tubule cells significantly increased the percentages of VASA positive cells (FIG. 10A), BOULE positive cells (FIG. 10B) and acrosin positive cells (FIG. 10C) after 4 weeks of culture compared to control medium.

### Example 6

### Conditioned Media of Sertoli Cell Cultures Induced Prepubertal Mice Spermatogonial Cells Differentiation In Vitro, Using Methylcellulose System, to Express High Levels of Acrosin and Protamine

Study question: Do Sertoli cells secrete factors that may induce proliferation and differentiation of mouse spermatogonial cells to meiotic and postmeiotic stages in vitro?

Summary answer: Addition of Sertoli cells conditioned media to spermatogonial cells in vitro, using methylcellulose culture system induced their development to cells that express acrosin and protamine.

What is known already: Sertoli cells (SCs) are active in the process of spermatogenesis and are in a direct contact with spermatogonial cells during their development from spermatogonial stem cells to sperm. Endocrine factors such as follicle stimulating hormone induce SCs to produce factors crucial for spermatogenesis. In addition, SPGCs are involved in the regulation of SC function. SCs produce different factors that affect the proliferation of the spermatogonial cells such as glial-derived nerve growth factor (GDNF), stem cell factor (SCF), leukemia inhibitory factor (LIF) and others. However, the factors that are involved in the differentiation of the spermatogonial cell are not yet clear.

Study design, size, and duration: Seven-days-old mice were used to isolate cells from the seminiferous tubules by 2-step enzymatic digestion. Sertoli cell cultures were prepared by culturing the STCs for 3 days in 37° C. in 5% CO₂ incubator. Thereafter, the cells were treated with hypotonic shock to eliminate the residual germ cells, and re-culture in DMEM media containing 10% KSR. After over-night incubation the media were replaced by fresh which was collected after 8 hours and stored at −80′C.

Participants/materials, setting, methods: Isolated cells from the seminiferous tubules (STCs) which contain spermatogonial cells were cultured in vitro in methylcellulose culture system which contained GDNF, LIF, SCF with or without conditioned media (CM) which was collected from SC cultures. The effect of this CM on the development of spermatogenesis in vitro was evaluated by examining the presence/expression of markers of premeiotic (VASA, GFR-alpha, SALL4), meiotic (BOULE) and postmeiotic (ACROSIN, PROTAMINE) stages by immunostaining or by qPCR analysis.

Main results and the role of chance: The results showed that isolated cells from seminiferous tubules of 7-days-old mice contained only premeiotic cells (VASA presence in around 8% of cells representing spermatogonial cells), but not meiotic or postmeiotic cells. These spermatogonial cells proliferated in vitro, in methylcellulose culture system, in the presence of GDNF, LIF and SCF to premeiotic (VASA presence in around 18% of cells), meiotic (BOULE in around 12% of cells) and postmeiotic cells (ACROSIN in around 8% of cells) after 4-5 weeks of culture (control system). Addition of 10% or 40% (v/v) of SC conditioned media to the spermatogonial cells in the culture did not significantly affect the proliferation of premeiotic cells compared to the control system. However, addition of SC conditioned media to the spermatogonial cells in vitro significantly increased their development leading to the presence of acrosin in around 20% of the cells and around 1.5 fold of increase in the RNA expression. Furthermore, an increase of about 3 fold in the RNA expression of protamine was found compared to the control system.

Wider implications of the findings: This is the first demonstration that SCs produce factors that could induce development of spermatogonial cells to postmeiotic cells in vitro. These differentiating factors present in SC conditioned media need to be identified. Hence, specific embodiments of the invention suggest using the in vitro system for male fertility preservation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

Additional references are cited in text
1. Valli, H. et al. Germline stem cells: toward the regeneration of Spermatogenesis. Fertility and sterility 101, 3-13 (2014).
2. Huleihel, M., Abuelhija, M. & Lunenfeld, E. In vitro culture of testicular germ cells: regulatory factors and limitations. Growth Factors 25, 236-252 (2007).
3. Huleihel, M., Nourashrafeddin, S. & Plant, T. M. Application of three-dimensional culture systems to study mammalian spermatogenesis, with an emphasis on the rhesus monkey (Macaca mulatta). Asian J Androl 17, 972-980 (2015).
4. Michailov, Y., Lunenfeld, E., Kapelushnik, J., Huleihel, M. Leukemia and male infertility: past, present, and future. Leuk Lymphoma 5, 1126-1135 (2019).
5. O'Shaughnessy, P. J. Hormonal control of germ cell development and Spermatogenesis, Seminars in cell& developmental biology 29, 55-56 (2014).
6. Plant, T. M., Marshall, G. R. The functional significance of FSH in spermatogenesis and the control of its secretion in male primates. Endocr Rev 22, 764-786 (2001).
7. Oduwole, O. O., Peltoketo, H., Huhtaniemi, I. T. Role of Follicle-Stimulating Hormone in Spermatogenesis. Front. Endocrinol (Lausanne) 9, 763 (2018).
8. Smith L B, Walker W H. The regulation of Spermatogenesis by androgens, In Seminars in cell & developmental biology, 30, 2-13, 2014.
9. Zeyse, D., Lunenfeld, E., Beck, M., Prinsloo, I., Huleihel, M. Interleukin-1 receptor antagonist is produced by Sertoli cells in vitro. Endocrinology 141, 1521-1527 (2000).
10. Huleihel, M., Lunenfeld, E. Regulation of spermatogenesis by paracrine/autocrine testicular factors. Asian J Androl 6, 259-68 (2004).
11. Loveland, K. L. et al. Cytokines in male fertility and reproductive pathologies: immunoregulation and beyond. Front Endocrinol 8, 1-16 (2017).
12. Huleihel, M., Lunenfeld, E. Involvement of intratesticular IL-1 system in the regulation of Sertoli cell functions. Molecular and cellular endocrinology 187, 125-132 (2002).
13. Rozwadowska, N., Fiszer, D., Jedrzejczak, P., Kosicki, W., Kurpisz, M. Interleukin-1 superfamily genes expression in normal or impaired human Spermatogenesis. Genes and immunity 8, 100 (2007).
14. Parvinen, M., Soder, O., Mali, P., Froysa, B., Ritzen, E. M. In vitro stimulation of stage-specific deoxyribonucleic acid synthesis in rat seminiferous tubule segments by interleukin-1 alpha. Endocrinology 129, 1614-20 (1991).
15. Pollanen, P., Soder, O., Parvinen, M. Interleukin-1 alpha stimulation of spermatogonial proliferation in vivo. Reprod Fertil Dev 1, 85-7 (1989).
16. O'Shaughnessy, P. J., Hu, L., Baker, P. J. Effect of germ cell depletion on levels of specific mRNA transcripts in mouse Sertoli cells and Leydig cells. Reproduction 135, 839-50 (2008).
17. Smith, L. B., O'Shaughnessy, P. J., Rebourcet, D. Cell-specific ablation in the testis: what have we learned?. Andrology 3, 1035-49 (2015).
18. Sam, A. D. et al. Sepsis produces depression of testosterone and steroidogenic acute regulatory (StAR) protein. Shock 11, 298-301 (1999).
19. Assmus, M. et al. Single subcutaneous administration of chorionic gonadotropin to rats induces a rapid and transient increase in testicular expression of pro-inflammatory cytokines. Pediatr. Res. 57, 896-901 (2005).
20. Mealy, K., Robinson, B., Millette, C. F., Majzoub, J., Wilmore, D. W. The testicular effects of tumor necrosis factor. Ann. Surg 211, 470-475 (1990).
21. van der Poll, T., Romijn, J. A., Endert, E., Sauerwein, H. P. Effects of tumor necrosis factor on the hypothalamic-pituitary-testicular axis in healthy men. Metabolism 42, 303-307 (1993).
22. De, S. et al. Expression of tumor necrosis factor-α in mouse spermatogenic cells. Endocrinology 133, 389-396 (1993).
23. Siu, M. K. Y., Lee, W. M., Cheng, C. Y. The interplay of collagen IV, tumor necrosis factor-α gelatinase B (matrix metalloprotease-9), and tissue inhibitor of metalloprotease-1 in the basal lamina regulates Sertoli cell-tight junction dynamics in the rat testis. Endocrinology 144, 371-387 (2003).
24. Baker, S. J., Reddy, E. P. Modulation of life and death by the TNF receptor superfamily. Oncogene 17, 3261-3270 (1998).
25. Pentiksinen, V. et al. TNF alpha down-regulates the Fas ligand and inhibits germ cell apoptosis in the human testis. J. Cli.n Endocrinol. Metab. 86, 4480-4488 (2001).
26. Suescun, M. O., Rival, C., Theas, M. S., Calandra, R. S., Lustig, L. Involvement of tumor necrosis factor-alpha in the pathogenesis of autoimmune orchitis in rats. Biol. Reprod 68, 2114-2121 (2003).
27. Schuppe, H. C. Chronic orchitis: a neglected cause of male infertility?. Andrologia 40, 84-91 (2008).
28. Theas, M. S. Tumor necrosis factor-α released by testicular macrophages induces apoptosis of germ cells in autoimmune orchitis. Human Reprod. 23, 1865-1872 (2008).
29. Lysiak, J. J. The role of tumor necrosis factor-alpha and interleukin-1 in the mammalian testes and their involvement in testicular torsion and autoimmune orchitis. Reproductive Biology and Endocrinology 2, 9 (2004).
30. Calabresi, P., Parks, R. Antiproliferative agents and drugs used for immunosuppression (ed. Goodman-Gillman, A., Goodman, L. S. & Gillman, A.) 1256-1306 (Macmillan, 1980).
31. Comish, P. B. Fetal cyclophosphamide exposure induces testicular cancer and reduced Spermatogenesis and ovarian follicle numbers in mice. PloS one 9, e93311 (2014).
32. Elangovan, N., Chiou, T. J., Tzeng, W. F., Chu, S. T. Cyclophosphamide treatment causes impairment of sperm and its fertilizing ability in mice. Toxicology 222, 60-70 (2006).
33. Codrington, A. M., Hales, B. F., Robaire, B. Spermiogenic germ cell phase-specific DNA damage following cyclophosphamide exposure. J Androl 25, 354-362 (2004).
34. Codrington, A. M., Hales, B. F., Robaire, B. Exposure of male rats to cyclophosphamide alters the chromatin structure and basic proteome in spermatozoa. Hum Reprod 22, 1431-1442 (2007).
35. Amon, J., Meirow, D., Lewis-Roness, H., Omoy, A. Genetic and teratogenic effects of cancer treatments on gametes and embryos. Hum Reprod Update 7, 394-403 (2001).
36. Liu, M., Hales, B. F., Robaire, B. Effects of four chemotherapeutic agents, bleomycin, etoposide, cisplatin, and cyclophosphamide, on DNA damage and telomeres in a mouse spermatogonial cell line. Biology of reproduction 90, 72-1 (2014).
37. Kanth, M. A., Kaur, P., Ahmad, B., Sharma, S. Histological effect of anticancer drug cyclophosphamide (CPA) on testes of Rattus. Indo American Journal of Pharmaceutical Research 4, 2645-2649 (2014).
38. Gassei, K., Orwig, K. E. Experimental methods to preserve male fertility and treat male factor infertility. Fertil Steril 105, 256-266 (2015).
39. Picton, H. M. et al. A European perspective on testicular tissue cryopreservation for fertility preservation in prepubertal, and adolescent boys. Hum Reprod 30, 2463-2475 (2015).
40. Huleihel, M. Spermatogenesis in an artificial three-dimensional system. Stem Cells 30, 2355-2360 (2012).
41. Jahnukainen, K., Stukenborg, J. B. Present and future prospects of male fertility preservation for children and adolescents. J Clin Endocrinol Metab 97, 4341-4351 (2012).
42. Loren, A. W. et al. Fertility preservation for patients with cancer: American Society of Clinical Oncology clinical practice guideline update. Journal of Clinical Oncology 31, 2500-2510 (2013).
43. Dobrinski, I., Travis, A. J. Germ cell transplantation for the propagation of companion animals, non-domestic and endangered species. Reproduction, Fertility and Development 19, 732-739 (2007).
44. Elhija, M. A., Lunenfeld, E., Schlatt, S., Huleihel, M. Differentiation of murine male germ cells to spermatozoa in a soft agar culture system. Asian journal of andrology 14, 285 (2012).
45. Stukenborg, J. B. et al. Coculture of Spermatogonia With Somatic Cells in a Novel Three-Dimensional Soft-Agar-Culture-System. Journal of andrology 29, 312-329 (2008).
46. AbuMadighem, A. et al. Development of Spermatogenesis In Vitro in Three-Dimensional Culture from Spermatogonial Cells of Busulfan-Treated Immature Mice. International Journal of Molecular Sciences 19, 3804 (2018).
47. Stukenborg, J. B. et al. New horizons for in vitro Spermatogenesis? An update on novel three-dimensional culture systems as tools for meiotic and postmeiotic differentiation of testicular germ cells. Molecular human reproduction 15, 521-529 (2009).
48. Abofoul-Azab, M. et al. Development of Postmeiotic Cells In Vitro from Spermatogonial Cells of Prepubertal Cancer Patients. Stem Cells Dev. 27, 1007-1020 (2018).
49. Chemes, H. E. Infancy is not a quiescent period of testicular development. International Journal of Andrology 24, 2-7 (2001).
50. Sharp, R. M., McKinnell, C., Kivlin, C., & Fischer, J. S. Proliferation and functional maturation of Sertoli cells, and their relevance to disorders of testis function in adulthood. Reproduction 125, 769-784 (2003).
51. Meistrich, M. L. Male Gonadal Toxicity. Pediatric Blood & Cancer 53, 261-266 (2009).
52. Gandini, L. et al. Effect of chemo- or radiotherapy on sperm parameters of testicular cancer patients. Hum Reprod 21, 2882-2889 (2006).
53. Anderson, R. A. et al. Cancer treatment and gonadal function: experimental and established strategies for fertility preservation in children and young adults. The Lancet Diabetes & Endocrinology 3, 556-567 (2015).
54. Cai, L., Hales, B. F. & Robaire, B. Induction of apoptosis in the germ cells of adult male rats after exposure to cyclophosphamide. Biol Reprod 56, 1490-1497 (1997).
55. Suter, L., Bobadilla, M., Koch, E. & Bechter, R. Flow cytometric evaluation of the effects of doxorubicin on rat spermatogenesis. Reprod Toxicol 11, 521-531 (1997).
56. Zhang, X., Yamamoto, N., Soramoto, S. & Takenaka, I. Cisplatin-induced germ cell apoptosis in mouse testes. Arch Androl 46, 43-49 (2001).
57. Seaman, F., Sawhney, P., Giammona, C. J. & Richburg, J. H. Cisplatin-induced pulse of germ cell apoptosis precedes long-term elevated apoptotic rates in C57/BU6 mouse testis. Apoptosis 8, 101-108 (2003).
58. Drumond, A. L. et al. Effects of multiple doses of cyclophosphamide on mouse testes: accessing the germ cells lost, and the functional damage of stem cells. Reprod Toxicol 32, 395-406 (2011).
59. Smart, E. et al. Chemotherapy drugs cyclophosphamide, cisplatin and doxorubicin induce germ cell loss in an in vitro model of the prepubertal testis. Sci Rep. 29, 1773 (2018).
60. Velez, D. L. C. J. et al. Reproductive effects of the anticancer drug cyclophosphamide in male rats at different ages. Arch Androl. 22, 251-63 (1989).
61. Zohni, K., Zhang, X., Tan, S. L., Chan, P. & Nagano, M. C. The efficiency of male fertility restoration is dependent on the recovery kinetics of spermatogonial stem cells after cytotoxic treatment with busulfan in mice. Hum Reprod 27, 44-53 (2012).
62. Akbarinejad, V., Tajik, P., Movahedin, M. & Youssefi, R. Effect of Removal of Spermatogonial Stem Cells (SSCs) from In Vitro Culture on Gene Expression of Niche Factors in Bovine. Avicenna J Med Biotechnol 8, 133-138 (2016).
63. Westernströer, B. et al. Profiling of Cxcl12 receptors, Cxcr4 and Cxcr7 in murine testis development and a spermatogenic depletion model indicates a role for Cxcr7 in controlling Cxcl12 activity. PLoS One. 9, e112598 (2014).
64. Garcia, T. X., Hofmann, M. C. Regulation of germ line stem cell homeostasis. Animal reproduction 12, 35 (2015).
65. França, L. R., Hess, R. A., Dufour, J. M., Hofmann, M. C., Griswold, M. D. The Sertoli cell: one hundred fifty years of beauty and plasticity. Andrology 4, 189-212 (2016).
66. Garcia, T. X., DeFalco, T., Capel, B., Hofmann, M. C. Constitutive activation of NOTCH1 signaling in Sertoli cells causes gonocyte exit from quiescence. Developmental biology 377, 188-201 (2013).
67. Garcia, T. X., Hofmann, M. C. NOTCH signaling in Sertoli cells regulates gonocyte fate. Cell Cycle 12, 2538-2545 (2013).
68. Garcia, T. X., Parekh, P., Gandhi, P., Sinha, K., Hofmann, M. C. The NOTCH Ligand JAG1 Regulates GDNF Expression in Sertoli Cells. Stem Cells Dev. 26, 585-598 (2017).
69. Galdon, G., Atala, A., Sadri-Ardekani, H. In Vitro Spermatogenesis: How Far from Clinical Application?, Curr Urol Rep 17, 49 (2016).
70. Komeya, M., Sato, T., Ogawa, T. In vitro spermatogenesis: A century-long research journey, still half way around. Reprod Med Biol 17, 407-420 (2018).
71. Suominen, J. S., Wang, Y., Kaipia, A., Toppari, J. Tumor necrosis factor-alpha (TNF alpha) promotes cell survival during Spermatogenesis, and this effect can be blocked by infliximab, a TNF-alpha antagonist. European Journal of Endocrinology 151, 629-640 (2004).
72. Abofoul-Azab, M. et al. Identification of Premeiotic, Meiotic, and Postmeiotic Cells in Testicular Biopsies Without Sperm from Sertoli Cell-Only Syndrome Patients. Int. J. Mol. Sci. 20, 470 (2019).

1. A method of in vitro proliferation and/or maturation of spermatogonium, comprising culturing said spermatogonium in a three-dimensional methylcellulose culture system (MCS) in a culture medium which comprises an effective concentration of a factor selected from the group consisting of granulocyte macrophages-colony stimulating factor (GM-CSF), interleukin-1 alpha (IL-1α), interleukin-1 beta (IL-1β) and interleukin-6 (IL-6), under conditions capable of inducing proliferation of said spermatogonium and/or differentiating said spermatogonium into at least meiotic and/or postmeiotic cells, thereby in vitro proliferating and/or maturing the spermatogonium.

2. The method of claim 1, further comprising culturing cells developed following a pre-determined time period from said spermatogonium in a second culture medium which comprises said factor and an effective concentration of testosterone.

3. A method of in vitro proliferation and/or maturation of spermatogonium, comprising culturing said spermatogonium in a three-dimensional methylcellulose culture system (MCS) in a culture medium which comprises an effective concentration of a granulocyte macrophages-colony stimulating factor (GM-CSF) and/or an effective concentration of interleukin-1 alpha (IL-1α) under conditions capable of inducing proliferation of said spermatogonium and/or differentiating said spermatogonium into at least meiotic and/or postmeiotic cells, thereby in vitro proliferating and/or maturing the spermatogonium.

4. The method of claim 3, further comprising culturing cells developed following a pre-determined time period from said spermatogonium in a second culture medium which comprises said GM-CSF and/or said IL-1α and an effective concentration of testosterone.

5. The method of claim 2, wherein said predetermined time period is two weeks±2 days.

6-7. (canceled)

8. The method of claim 1, wherein said culture medium further comprises an effective concentration of at least one growth factor selected from the group consisting of Glial cell line-derived neurotrophic factor (GDNF), leukemia inhibitory factor (LIF), basic fibroblast growth factor (bFGF), and epidermal growth factor (EGF).

9. (canceled)

10. The method of claim 1, wherein said culture medium comprises serum replacement.

11. (canceled)

12. The method of claim 1, wherein said culture medium comprises STEM PRO® (Thermo Fisher Scientific) supplement.

13. (canceled)

14. The method of claim 1, wherein said culture medium further comprises a Sertoli cell conditioned medium.

15. A method of in vitro proliferation and/or maturation of spermatogonium, comprising culturing said spermatogonium in a three-dimensional methylcellulose culture system (MCS) in a culture medium which comprises Sertoli cells conditioned medium under conditions capable of inducing proliferation of said spermatogonium and/or differentiating said spermatogonium into at least meiotic and/or postmeiotic cells, thereby in vitro proliferating and/or maturing the spermatogonium.

16. The method of claim 15, wherein said culture medium further comprises at least one growth factor selected from the group consisting of: Glial cell line-derived neurotrophic factor (GDNF), leukemia inhibitory factor (LIF), and stem cell factor (SCF).

17. The method of claim 15, wherein said culture medium further comprises an effective concentration of a factor selected from the group consisting of granulocyte macrophages-colony stimulating factor (GM-CSF), interleukin-1 alpha (IL-1α), interleukin-1 beta (IL-1β) and interleukin-6 (IL-6).

18-19. (canceled)

20. The method of claim 15, wherein said Sertoli condition medium comprises of 10%-40% (volume/volume) of the culture medium.

21. The method of claim 1, wherein said spermatogonium is comprised in a testicular biopsy of a subject.

22. (canceled)

23. The method of claim 1, wherein said spermatogonium is of a subject who is infertile or at risk of becoming infertile.

24. (canceled)

25. The method of claim 1, wherein said spermatogonium if of a subject who is a prepubertal subject.

26. The method of claim 1, wherein said spermatogonium if of a subject in need of aggressive chemotherapy and/or aggressive radiotherapy.

27-29. (canceled)

30. The method of claim 1, wherein said spermatogonium is of a subject in need of bone marrow transplantation.

31. The method of claim 1, wherein said spermatogonium is of a subject inflicted with cancer associated with male infertility.

32-38. (canceled)

39. A cell obtainable according to the method of claim 1, wherein said cell is characterized by at least the expression of acrosin.

40. (canceled)
